(19) United States
(12) Patent Application Publication
(10) Pub. No.: US 2007/0250942 A1
(43) Pub. Date: Oct. 25, 2007

(54) Non-human transgenic mammals useful for identifying and assessing neural stem/progenitor cells

(75) Inventors: Grigori Enikolopov, Cold Spring Harbor, NY (US); Juan Manuel Encinas, East Northport, NY (US)

Correspondence Address: FISH & NEAVE IP GROUP;ROPES & GRAY LLP, 1211 AVENUE OF THE AMERICAS, NEW YORK, NY, 10036-8704, US

(73) Assignee: Cold Spring Harbor Laboratory, Cold Spring Harbor, NY (US)

(21) Appl. No.: 11/784,523

(22) Filed: Apr. 6, 2007

Related U.S. Application Data
(60) Provisional application No. 60/790,387, filed on Apr. 7, 2006.

(51) Int. Cl. A01K 67/027

(52) U.S. Cl. 800/18

(57) ABSTRACT

Non-human transgenic mammals are produced which have, incorporated in their genome, DNA which includes a regulatory sequence of a mammalian nestin gene, operably linked to a gene coding for a nuclear localization signal peptide fused to a marker protein or reporter protein. The regulatory sequence can include a promoter and a sequence present in the second intron of the mammalian nestin gene. Preferably, the marker protein or reporter protein is a fluorescent protein, for example a cyan fluorescent protein, modified for enhanced fluorescence. Multipotent and, in particular, neural stem and progenitor cell populations are quantitatively observed in the organs of the non-human transgenic mammal or progeny thereof. Multipotent stem and progenitor cells are isolated directly from the non-human transgenic mammal, progeny or embryo thereof, for example by FACS, without culture passages.

### CROSS-REFERENCE TO RELATED APPLICATION

This application claims priority to U.S. Provisional Application Ser. No. 60/790,387 filed Apr. 7, 2006, the entire content of which is incorporated by reference.

### TECHNICAL FIELD OF INVENTION

Non-human transgenic mammals are produced which have, incorporated in their genome, DNA which includes a regulatory sequence of a mammalian nestin gene, operably linked to a gene coding for a nuclear localization signal peptide fused to a marker or reporter protein. The regulatory sequence can include a promoter and a sequence present in the second intron of the mammalian nestin gene. Preferably, the marker or reporter protein is a fluorescent protein, for example a cyan fluorescent protein, modified for enhanced fluorescence. Multipotent and, in particular, neural stem and progenitor cell populations are observed in the organs of the non-human transgenic mammal or progeny thereof. Multipotent stem and progenitor cells are isolated directly from the non-human transgenic mammal, progeny or embryo thereof, for example by FACS, without culture passages.

### BACKGROUND OF THE INVENTION

Critical features of neuropathology and/or the effective targets of therapeutic treatment may be limited to a subpopulation of neuronal cells in a particular stage within the neuronal proliferation-differentiation cascade. Particular targets (e.g., stem cells vs. early progenitors vs. advanced neuroblasts) may imply different molecular mechanisms of controlling cell division and survival, different circuits affected by a given drug, and different insights on the behavioral action of a given drug. Furthermore, dissimilar cellular mechanisms of a drug action in early-stage vs. late-stage precursor cells may result in different effects of a given drug on mature vs. juvenile brain, because the latter consists of a much larger number of early-stage precursor cells. The possibility of such different effects raises concerns regarding the use of a drug targeting neuronal cells in children, even when that drug has been tested and considered safe and effective in adults.

For example, antidepressant drugs of the selective serotonin reuptake inhibitor (SSRI) class (e.g., fluoxetine) are commonly used to treat a wide spectrum of mood disorders in adults (M. L. Wong and J. Licinio, Nat. Rev. Neurosci. 2, 343 (2001)). They are also increasingly prescribed to children and adolescents (C. J. Whittington et al., Lancet 363, 1341 (2004); N. D. Ryan, Lancet 366, 933 (2005)). Numerous long-term studies have demonstrated the efficacy and safety of SSRI antidepressants for adult patients (J. F. Wernicke, Expert Opin. Drug Saf. 3, 495 (2004); J. Licinio and M. L. Wong, Nat. Rev. Drug Discov. 4, 165 (2005)). However, concerns remain regarding the use of SSRIs in children (Whittington, above; Ryan, above; B. Vitiello and S. Swedo, New Eng. J. Med. 350, 1489 (2004); I. C. Wong et al., Drug Saf 27, 991 (2004)) and the risk of adverse effects of such antidepressants during the course of treatment or even later in life.

SSRI fluoxetine increases generation of new neurons in the dentate gyrus (DG) of the adult brain (J. E. Malberg et al., J Neurosci 20, 9104 (2000); B. L. Jacobs et al., Mol. Psychiatry 5, 262 (2000); J. E. Malberg and R. S. Duman, Neuropsychopharmacol. 28, 1562 (2003); L. Santarelli et al., Science 301, 805 (2003); D. C. Lie et al., Annu. Rev. Pharmacol. Toxicol. 44, 399 (2004); R. S. Duman, Biol Psychiatry 56, 140 (2004)). Importantly, recent findings suggest that this increase may be a causative factor in the behavioral effects of this class of antidepressants (Santarelli, above). These discoveries to define the cellular basis for the action of SSRIs are important advances which may provide a novel framework for understanding depression and designing new therapeutic drugs. However, the steps within the neuronal differentiation cascade (Lie et al, above; G. Kempermann et al., Trends Neurosci 27, 447 (2004); Seri et al., J. Comp. Neurol. 478, 359 (2004)) targeted by SSRIs remain unknown. Meanwhile, it remains critical to determine if SSRIs act similarly in the juvenile and adult brain. The possibility that antidepressants may act differently in the young and adult brains has raised medical and social concerns regarding the use of SSRI drugs in children and adolescents (C. J. Whittington et al., Lancet 363, 1341 (2004), N. D. Ryan, Lancet 366, 933 (2005), B. Vitiello & S. Swedo, New Eng. J. Med. 350, 1489 (2004), I. C. Wong et al. Drug Saf. 27, 991 (2004)). These concerns are heightened when considered with animal data, which show that exposure of newborn (p4) mice to fluoxetine results in elevated anxiety when these animals become adults (M. S. Ansorge, et al., Science 306, 879 (2004)).

However, the investigation of neuropathology and definition of targets of therapeutic treatment is hampered by the imprecision in identifying and quantifying the changes in each class of neural precursor cells in the brain. Accurate enumeration of changes in distinct subpopulations of neuronal precursors by immunocytochemistry is problematic: high cell density, complex cell morphology, and uncertainties in defining distinct boundaries between subclasses of cells reduces the precision of evaluating changes in particular subclasses of neuronal precursors (e.g., in contrast to 5-bromo-2-deoxyuridine (BrdU)- or thymidine-labeling of cell nuclei, where great precision can be achieved); this problem is particularly acute in the juvenile brain, where the number of neural stem and progenitor cells is particularly high. Similarly, currently available functional in vitro assays for identifying neural stem and progenitor cells (e.g., formation of neurospheres) do not provide confident measures of changes induced by a therapeutic agent on a cell-by-cell basis, because such an agent may be an inducer of neurogenesis that often results in a 30-40% increase in the number of newly generated cells. Furthermore, such assays presently cannot be performed for small subregions of neurogenic areas.

Transgenic mammals expressing a reporter gene controlled by a nestin regulatory region have been described previously (U.S. App. Pub. No. 2002/0178460 and J. L. Mignone et al., J. Comp. Neurol. 469, 311 (2004)). In such mammals, a reporter protein is detected generally in various compartments or the entirety of the cytosol of a cell expressing the reporter protein. With such a reporter protein, the number of cells expressing the reporter protein may be difficult to quantitatively determine, especially for cells whose morphology is not compact.

Therefore, there exists a need for a means to identify and quantitatively assess changes of the number of cells in the stem/progenitor cell compartment of a brain.

### SUMMARY OF THE INVENTION

The invention relates to a novel non-human transgenic mammal or its progeny or embryo, which mammal has integrated into its genome a reporter gene characterized by a nuclear localization signal operably linked to the regulatory region of a mammalian nestin gene. The reporter is expressed and translocated to nuclei in multipotent stem cells and progenitor cells of such a transgenic mammal, but not in further differentiated cells. In one embodiment, the stem and progenitor cells are neural stem and progenitor cells. According to the present invention, the reporter is localized to nuclei, thus allowing quantitative analysis of the number of cells expressing the reporter gene. In various embodiments of the invention, the reporter gene is selectively expressed in neural stem cells and progenitor cells.

According to an embodiment, this invention permits the assessment of the neuronal differentiation cascade, which comprises several clearly distinguishable steps based on expression of certain marker genes characteristic of the differentiation stage of the neuronal cells.

One embodiment of the invention is an expression construct comprising a mammalian nestin gene operably linked to a reporter gene, which reporter comprises a detectable polypeptide with a nuclear localization signal. In one embodiment, the expression construct further comprises a regulatory sequence found in the second intron of a mammalian nestin gene, which sequence is operably located relative to other elements of the expression construct. Another embodiment of the invention is a cell comprising such expression construct.

A non-human transgenic mammal according to this invention may be produced by: introducing into a fertilized egg of a non-human mammal DNA comprising a regulatory sequence of a mammalian nestin gene operably linked to a reporter gene, wherein said reporter gene comprises a sequence of a nuclear localization signal fused in-frame to a sequence encoding a detectable polypeptide; introducing such fertilized egg into an oviduct of a non-human mammal of the same species as the source of the fertilized egg to allow the fertilized egg to develop into a viable transgenic mammal; and selecting a non-human transgenic mammal that expresses said reporter which is translocated to nuclei of multipotent stem cells and progenitor cells. Another embodiment of this invention relates to an isolated stem or progenitor cell from a non-human transgenic mammal, the genome of which has integrated into it DNA comprising a regulatory sequence of a mammalian nestin gene operably linked to a reporter gene which comprises a sequence encoding a detectable polypeptide with a nuclear localization signal, wherein the reporter is expressed and translocated to nuclei in such a cell.

The novel non-human transgenic mammals of this invention allow quantitative assessment of changes in the stem/progenitor cell compartment of an organ, for example the brain, or a region of an organ. Thus, another embodiment of the invention relates to a method of quantitatively measuring the population of multipotent stem cells and/or the progenitor cells. In one embodiment, such a method comprises measuring a signal from a detectable reporter expressed in cells from an organ or a region of an organ of a non-human transgenic mammal which has integrated into its genome DNA a reporter gene with a nuclear localization signal operably linked to the regulatory region of a mammalian nestin gene, wherein the reporter is expressed and translocated to nuclei in multipotent stem cells and progenitor cells in the transgenic mammal. In such cells, the quantity of said signal correlates with the size of said population measured.

A further aspect of the invention relates to a method for assessing an effect of a compound on proliferation or differentiation of multipotent stem cells or progenitor cells. One embodiment of such a method comprises the steps of: in a test sample, contacting a compound with live multipotent stem cells or progenitor cells, the genome of which have integrated into it DNA comprising a regulatory sequence of a mammalian nestin gene operably linked to a reporter gene comprising a sequence encoding a detectable polypeptide with a nuclear localization signal, wherein the reporter is expressed and translocated to nuclei in such a cell; measuring a signal from the reporter in the presence of the compound; and comparing the signal to that of a relevant control sample. The difference between the signals for the test sample and the control sample, if any, indicates the compound's effect on proliferation or differentiation of the multipotent stem cells or progenitor cells.

### BRIEF DESCRIPTION OF THE DRAWINGS

FIG. 1A-Q shows a neuronal differentiation cascade in the dentate gyrus.

FIG. 2A-I shows the effect of fluoxetine on cell proliferation in the adult dentate gyrus.

FIG. 3A-G shows the effect of fluoxetine on the number of NB1 cells in the adult dentate gyrus.

FIG. 4A-H shows the effect of fluoxetine on proliferation of ANP cells in the adult dentate gyrus.

FIG. 5A-E shows the lack of effect of fluoxetine on neurogenesis in the subventricular zone SVZ.

FIG. 6A-J shows the effect of fluoxetine on neurogenesis in the adult dentate gyrus (30 day survival experiments).

FIG. 7A-M shows the effect of fluoxetine on proliferation of ANP cells in the juvenile dentate gyrus.

FIG. 8A-F shows the effect of fluoxetine on neurogenesis in the juvenile dentate gyrus (30 day survival experiments).

### DETAILED DESCRIPTION OF THE INVENTION

As used herein, the term “non-human transgenic mammal” includes the newly born, young offsprings, developing adults, or embryos of the non-human transgenic mammal, as well as newly born, young offsprings, developing adults or embryos of a progeny of the non-human transgenic mammal. Examples of non-human transgenic mammals and their progeny include mouse, rat, dog, monkey, as well as any other suitable non-human mammalian species. A preferred mammal is a rodent, including a mouse.

As used herein, the term “compound” includes, for example, pharmaceutical compounds, such as drugs and other biologically active compounds that may be administered in the treatment or prophylaxis of various medical indications or conditions. Such compounds are generally referred to herein as “therapeutic agents”. The term compound also includes pharmaceutical compounds that may be useful in diagnosis of various medical conditions or disorders. Disorders include diseases. Such compounds are generally referred to herein as “diagnostic agents.”

As used herein, the phrase “multipotent stem and progenitor cells” are cells which are capable of proliferating and differentiating into several possible cell types. Generally, a stem cell is thought of as a cell having the capacity to divide asymmetrically, producing one copy of itself and one, more committed daughter cell. Often, stem cells are thought of as undifferentiated cells with the ability to proliferate, to exhibit self-maintenance, to generate a large number of progeny and to generate new cells in response to injury or disease. Generally, a progenitor cell is a more committed cell which divides symmetrically and can be differentiated into more mature morphotypes. Generally, multipotent stem and progenitor cells have regional specificity and are capable, upon differentiation, of generating cell types characteristic of a certain organ or tissue present in a mammalian organism. Neural stem and progenitor cells are one example of multipotent stem and progenitor cells. These neural stem and progenitor cells are characterized in part by the expression of nestin. Upon differentiation, neural stem and progenitor cells give rise to cells with distinct functions, such as glial cells and neurons.

Embryonic or totipotent precursors of multipotent stem and progenitor cells are referred to herein as “totipotent stem and progenitor cells.” Due to their totipotent character, these cells are capable of differentiating into cells characteristic of any organ or tissue in a mammalian organism. As used herein, totipotent stem and progenitor cells are precursors of multipotent cells, do not possess regional specificity and can be distinguished from multipotent stem and progenitor cells by the fact that they do not express certain marker proteins. In neural cells, such a marker can be nestin.

As used herein, “a regulatory sequence of a mammalian nestin gene” includes one or more regulatory sequences of the nestin gene which, when operably linked to a gene encoding a protein, expresses the protein in multipotent stem and progenitor cells. In a particular embodiment, the regulatory sequence of nestin comprises the second intron sequence of a mammalian nestin gene.

Nestin is an intermediate filament protein; in particular, it defines a distinct class of intermediate filament protein. A variety of nestin genes or sequences thereof can be used in the products and methods of the present invention. Examples of suitable mammalian nestin genes include: the rat nestin gene, human nestin gene, mouse nestin gene and nestin genes specific to any other mammalian species. In a preferred embodiment of the invention, the mammalian nestin gene is the rat nestin gene.

Nestin genes of mammalian origin have been isolated and sequenced. For example, nucleotide sequences of rat and human nestin genes and deduced amino acid sequences of the corresponding nestin proteins are disclosed in U.S. Pat. No. 5,338,839 issued on Aug. 16, 1994 to McKay, et al., which is incorporated herein by reference in its entirety. Regulatory elements of the nestin gene, e.g., rat, are discussed, for example, in Zimmerman, L. et al., Neuron, 12, 11-24 (1994), which is incorporated herein by reference in its entirety.

Nestin is expressed, for example, in neural stem and progenitor cells. Its expression diminishes as neural stem and progenitor cells differentiate into neural cell types. In healthy mammals, fully differentiated cells of the CNS, such as neurons, astrocytes and oligodentrocytes, do not generally express nestin. However, nestin expression has been identified in some CNS tumors and after injury to the adult spinal cord or optic nerve. In the case of injury, nestin production has been observed in reactive astrocytes and in cells close to the central canal in the spinal cord. It has been reported (C. B. Johansson et al., Cell, 96, 25-34 (1999)) that, in adult mammals, cavity lining cells, such as ependymal cells, express nestin, in particular following spinal cord injury.

Nestin expression also has been observed in multipotent stem and progenitor cells other than neural stem and progenitor cells. As reported, for example, by Kobayashi, M., et al., Pediatr. Res. 43(3), 386-392 (1998), nestin is expressed in muscle precursors; however, mature muscle cells do not express nestin (Zimmerman, L. et al., above). Nestin expression has also been linked to developing organs such as, for example, the liver (Niki, T. et al., Hepatology 29(2), 520-527 (1999)), tooth (Terling, C. et al., Int. J. Dev. Biol. 39(6), 947-956 (1995), and heart (Kachinsky, A. M. et al., J. Histochem. Cytochem. 43(8), 843-847 (1995). In addition, nestin expression can occur in multipotent stem and progenitor cells of the pancreas, intestinal tract, and retina.

In one embodiment of the invention, the non-human transgenic mammal or progeny or embryo thereof has integrated into its genome DNA including a regulatory sequence of a mammalian nestin gene, wherein the regulatory sequence is such that a marker protein or a reporter protein that is operably linked to the regulatory sequence is expressed in multipotent stem and progenitor cells. In another embodiment of the invention, the regulatory sequence is such that a marker protein or a reporter protein is selectively expressed in multipotent stem and progenitor cells (e.g., the central nervous system). In yet another embodiment, the regulatory sequence selectively direct expression of a marker protein or a reporter protein in neural stem and progenitor cells.

In a preferred embodiment, the nestin regulatory sequence includes the entire second intron sequence of the mammalian nestin gene. Shorter sequences of the second intron also can be employed. Examples of suitable shorter sequences are known in the art. For example, in Eur. J. Neurosci., 9, 452-462 (1997), hereby incorporated by reference in its entirety, Lothian and Lendahl, showed that transgenic mice generated with the most conserved 714 bp in the 3′ portion of the second human intron or with the complete, 1852 bp, human intron gave very similar nestin-like expression pattern and concluded that the important control elements reside in the 714 bp element. In Exper. Cell Res., 248(2), 509-519 (1999), hereby incorporated by reference in its entirety, Lothian, et al. showed that a 374-bp region in the second intron of the human nestin gene is sufficient and a 120-bp sequence in this region is required for the expression of the nestin gene in neural cells of the embryonic CNS.

Optionally, the regulatory sequence can further include elements present in the first intron of the mammalian nestin gene. The entire sequence of the first intron or shorter sequences thereof can be employed. As discussed by Zimmerman, et al. above, hereby incorporated by reference in its entirety, independent and cell-type specific elements in the first and second introns of the nestin gene direct reporter gene expression to the developing muscle and neural precursors, respectively.

The regulatory sequence of a mammalian nestin gene, as defined herein, can include any suitable promoter. In one embodiment, the promoter can be a nestin promoter. In a preferred embodiment, the nestin promoter is obtained from the same mammalian nestin gene as the regulatory sequence. Suitable promoters also include promoter sequences which are functional in mammalian cells, yeast, bacteria or insect cells. Examples of suitable promoters include but are not limited to, polyhedrin, 3-phosphoglycerate kinase, metallothionein, retroviral LTR, SV40 and TK promoters and others known in the art.

In the products, compositions and methods of the present invention, the regulatory sequence of a mammalian nestin gene, as defined above, is operably linked to a gene coding for a marker protein or a reporter protein. The gene coding for the marker protein or reporter protein is expressed in multipotent stem and progenitor cells of the non-human transgenic mammal, progeny or embryo thereof. In one embodiment of the invention, the marker protein or reporter protein is selectively expressed in multipotent stem and progenitor cells. As used herein, the term “selectively expressed” means that the marker protein or reporter protein is expressed to a detectable level predominantly in multipotent stem and progenitor cells. In another embodiment, the marker protein or reporter protein is expressed to a detectable level in neural stem and progenitor cells. In yet another embodiment, the marker protein or reporter protein is selectively expressed in neural stem and progenitor cells.

In various embodiments of the invention, the nestin regulatory sequence is operatively linked to a sequence encoding a nuclear localization signal peptide, fused in-frame to a marker or reporter protein sequence. Marker proteins or reporter proteins for use in the products, compositions and methods of the present invention are known to those of skill in the art. Marker protein or reporter proteins for which there are convenient and simple assay methods are preferred. Examples include, but are not limited to, luminescent proteins, fluorescent proteins, enzymes, cell surface proteins and other marker or reporter proteins known in the art.

A preferred marker protein or reporter protein which can be employed is a fluorescent protein (FP). Examples of suitable fluorescent proteins include, but are not limited to, cyan fluorescent protein (CFP), green fluorescent protein (GFP), modified or enhanced green fluorescent protein (EGFP), yellow fluorescent protein, blue FP, red FP and their enhanced versions (Clontech) and any other luminscent or fluorescent protein that can emit light. In a preferred embodiment, the marker protein or reporter protein is a fluorescent protein, such as cyan fluorescent protein (CFP). In another, the CFP is modified for enhanced fluorescence. CFP as well as mutants of CFP are known to those skilled in the art. Green fluorescent proteins (GFPs) are also known in the art and are used extensively. For example, proteins exhibiting green fluorescence are described in U.S. Pat. No. 5,491,084 and in U.S. Pat. No. 5,804,387, which are incorporated herein by reference in their entirety. In still another embodiment of the invention, the fluorescent protein modified for enhanced fluorescence is enhanced green fluorescent protein (EGFP) which can be obtained from the pEGFP-N1 plasmid supplied by Clontech. Briefly, the plasmid included 190 silent base changes from human codon preferences; there was a conversion of the ATG codon for better Kozak consensus and amino acid substitutions: Phe64-Leu and Ser65-Thr.

The invention also relates to a method for producing a non-human transgenic mammal which expresses a fluorescent protein in multipotent stem and progenitor cells, comprising introducing into a fertilized egg of a non-human mammal, DNA comprising a regulatory sequence of a mammalian nestin gene, such as described above, operably linked to a gene coding for a nuclear localization signal peptide fused to a fluorescent protein, such as described above, that is expressed in multipotent stem and progenitor cells of the non-human mammal. The fertilized egg is introduced into a non-human mammal, preferably of the same species as the egg donor, to produce a non-human transgenic mammal and that mammal is allowed to produce progeny which are non-human transgenic mammal progeny. The method also comprises selecting from the non-human transgenic mammal progeny those progeny whose multipotent stem and progenitor cells express the fluorescent gene. In one embodiment, the method selects a non-human transgenic mammal whose neural stem and progenitor cells express the fluorescent gene. Genes expressing CFP or CFP modified for enhanced fluorescence, e.g., ECFP, are preferred.

Another aspect of the invention relates to a non-human transgenic mammal which expresses a fluorescent protein in stem and progenitor cells produced by a method comprising introducing into a fertilized egg of a non-human mammal, DNA comprising a regulatory sequence of a mammalian nestin gene, as defined above, operably linked to a gene coding for a fluorescent protein, such as described above, that is expressed in stem and progenitor cells of the non-human mammal; by introducing the fertilized egg into a non-human mammal, preferably of the same species as the egg donor, to produce a non-human transgenic mammal and that mammal is allowed to produce progeny which are non-human transgenic mammal progeny. From the non-human transgenic mammal progeny are selected those progeny whose stem and progenitor cells express the fluorescent gene. In one embodiment, the non-human transgenic mammal is the mammal whose neural stem and progenitor cells express the fluorescent gene.

In a preferred embodiment, the DNA comprising a regulatory sequence of a mammalian nestin gene operably linked to a nuclear localization signal peptide fused to a fluorescent protein is an expression construct or vector which comprises a promoter sequence, preferably a promoter sequence of a mammalian nestin gene, a gene coding for CFP and a regulatory sequence present in the second intron of a nestin gene. An example of such an expression construct, methods for producing such a construct, as well as methods for introducing the construct into the fertilized egg of the non-human mammal are further described below.

A cell or cells which comprise(s) the expression construct according to this invention can be isolated from the non-human transgenic mammal of this invention and further employed. For instance, such cells can be studied and characterized in vitro or can be used in experiments designed to monitor cellular development and/or differentiation. The marker protein or reporter protein is localized in the nuclei of such cells; thus making the observation of cellular behavior easier by locating each cell clearly separately, rather than as a general tangle of extended cell structures. Cells which comprise an expression construct of the invention also can be transplanted into organs of recipient animals, including mammals. Such cells may be employed in other clinical, diagnostic, laboratory and experimental methods known in the art.

The invention also relates to assessing the presence and quantity of multipotent stem and progenitor cells in the organism, organs or a region thereof, of the non-human transgenic mammal, in its progeny or in the non-human transgenic embryo of the invention. In a preferred embodiment, the non-human transgenic mammal employed has integrated into its genome DNA comprising a regulatory sequence of a mammalian nestin gene operably linked to a gene coding for a nuclear localization signal peptide fused to a fluorescent protein. Populations of multipotent stem and progenitor cells can be assessed by viewing or measuring fluorescence from an organ or region thereof of the non-human transgenic mammal, progeny or embryo thereof, and counting the nucleus that contain the reporter protein. The presence of fluorescent cells also can be assessed in organs subjected to trauma, during tissue or organ regeneration, during various treatments, before and after transplantation, and during various stages of development, in the presence or absence of various environmental factors or stimuli. In vivo effects of compounds administered to animal models and affecting multipotent stem and progenitor cells can be evaluated by using the non-human transgenic mammal of the invention and by measuring the fluorescence of an organ or region thereof and comparing it to the fluorescence of the organ or region thereof in control animals.

Another aspect of the invention also relates to a method for obtaining or isolating primary, non-cultured multipotent (e.g., neural) stem and progenitor cells. Such cells are also referred to herein as intact, fresh, or simply primary multipotent stem and progenitor cells. Such cells can be and are obtained from a non-human transgenic mammal of the invention, from a progeny thereof or from a non-human transgenic mammalian embryo, directly, without culture passages. However, the use of intact, fresh, or primary multipotent stem and progenitor cells isolated from a non-human transgenic mammal of the invention are not limited to direct use of the cells without further cultivation; such cells may also be used for in vitro studies. Accordingly, once obtained from the non-human transgenic mammal or progeny thereof, the primary multipotent stem and progenitor cells isolated according to the method of the invention can be further cultivated in vitro using techniques known by those skilled in the art.

Such a method of obtaining live primary multipotent stem and progenitor cells comprises isolating cells which express the marker protein or reporter protein defined above from a non-human transgenic mammal, progeny or embryo thereof, which has integrated into its genome DNA comprising a regulatory sequence of a mammalian nestin gene operably linked to a gene coding for a nuclear localization signal peptide fused to a marker protein or reporter protein, wherein the gene coding for the marker protein or reporter protein is expressed in multipotent stem and progenitor cells, and wherein the marker protein or reporter protein is translocated into the nuclei of such cells of the non-human transgenic mammal, progeny or embryo thereof. Another method of obtaining live primary multipotent stem and progenitor cells comprises isolating fluorescent cells from a non-human transgenic mammal, progeny or embryo thereof, which has integrated into its genome DNA comprising a regulatory sequence of a mammalian nestin gene operably linked to a gene coding for a nuclear localization signal peptide fused to a fluorescent protein, wherein the gene coding for the fluorescent protein is expressed in multipotent stem and progenitor cells, and wherein the fluorescent protein is translocated into the nuclei of such cells of the non-human transgenic mammal, progeny or embryo thereof.

Multipotent stem and progenitor cells present in organs or regions thereof can be isolated by the products, compositions and methods of the invention. In a preferred embodiment, the isolated cells are neural stem and progenitor cells. Multipotent stem and progenitor cells present in other organs and expressing nestin, for example muscle precursor cells, can also be purified (e.g., highly enriched).

In a preferred embodiment, cells expressing a fluorescent protein can be isolated using fluorescent activated cell sorting (FACS). In the case of proteins modified for enhanced fluorescence, the brightness of the transgene-expressing cells is very high and FACS proves to be a quick and efficient procedure. FACS techniques are known to those skilled in the art. The use of FACS for sorting cells is discussed, for example, in U.S. Pat. No. 5,804,387. In a preferred embodiment, the fluorescent protein is green fluorescent protein enhanced for fluorescence and identified as EGFP. Primary, non-cultured EGFP expressing cells can be isolated from the intact organism by FACS in less than an hour, typically in 10 to 30 minutes.

Other methods can be employed for obtaining or isolating cells which have integrated in their genome DNA comprising a regulatory sequence of a mammalian nestin gene operably linked to the sequence encoding a nuclear localization signal peptide fused to a marker protein or reporter protein. Examples include, but are not limited to, using the fluorescent (Herzberg) β-galactose substrate as described by Nolan, G. P., et al., Proc. Natl. Acad. Sci. USA 85(8), 2603-2607 (1988) or the method described by Stemple, D. L., et al., Cell, 71(6), 973-85 (1992).

Once isolated, reporter-expressing cells can be further studied and characterized by techniques known to those skilled in the art. In one embodiment of the invention, RNA and proteins are separated from isolated primary cells. Proteins specific to the isolated cells can be identified, for example, by two dimensional electrophoresis or by isoelectrofocusing.

In another embodiment of the invention, genes characterizing the intact cells, isolated as described above, are identified as well. For example, this can be accomplished by versions of gene chip technology. Examples of gene chip approaches known to those skilled in the art include the Affimetrix or Synteni approaches. One procedure for identifying such genes includes preparing a catalog or library of, for example, genes, cDNA, expressed sequence tags (EST) in the isolated cells and comparing the catalog against genes expressed in non-fluorescent cells. The non-fluorescent cells may be cells that are in an earlier stage of development, (e.g., totipotent cells) than cells that are in the nestin-expressing stage or may be cells that have differentiated beyond the nestin-expressing stage. Similarly, the catalog can be compared to genes expressed by non-fluorescent cells in specific organs or regions thereof.

In still another embodiment of the invention, surface antigens specific to the cells isolated as described above are identified. Techniques for identifying surface cell specific surface antigens are known to those skilled in the art. These techniques include, for example, immunizing animals with the isolated cells and obtaining antibodies directed against cell specific antigens from the immunized animals.

In yet another embodiment of the invention, cells isolated according to the invention are transplanted into animals. In particular, the isolated cells can be transplanted into specific organs or regions thereof. Techniques for accomplishing the transplantation of isolated cells into an animal are known to those skilled in the art. The animal may be of the same species as the non-human transgenic mammal of the invention. Alternatively, the animal can be of a different species. Examples of animals include mammals, such as rodents including, e.g., a mouse, a rabbit, or a rat; primates including, e.g., a monkey; canines, including, e.g., a dog; ovines including, e.g., a sheep; equines, including, e.g., a horse, and many others.

The non-human transgenic mammal or progeny or embryo thereof described above and cells isolated according to the invention can be employed to identify compounds that affect the differentiation of totipotent and multipotent stem and progenitor cells. Preferred therapeutic agents include growth factors and neutrophins. Other compounds which can be employed include but are not limited to: small molecules (such as organic or organometallic molecules), vitamins, proteins, peptides, polypeptides, viruses, nucleic acids, hormones (such as growth factors), enzymes (for example, nitric oxide synthase), and other biological compounds of natural or recombinant DNA origin which may be implicated in cellular development or differentiation.

As described herein, the present invention further relates to methods of identifying whether a compound (i) promotes multipotent stem and progenitor cell differentiation; (ii) is toxic to multipotent stem and progenitor cells; (iii) promotes differentiation of totipotent to multipotent stem and progenitor cells; or (iv) promotes differentiation of multipotent stem and progenitor cells into neural cells. The methods include detecting or measuring the expression of a marker protein or reporter protein. Methods of detecting or measuring marker protein or reporter gene expression are known to those of skill in the art. Luminescence, fluorescence, enzymatic activity (e.g. P-galactosidase), magnetic beads and other methods of antibody-based purification, fluorescent activated cell sorting, differential centrifugation and other assay methods, known in the art can be employed. A preferred marker protein or reporter gene is one which expresses a fluorescent protein, as described above. Fluorescence is measured by techniques and equipment known to those skilled in the art. Excitation and emission wavelengths are selected in accordance to the fluorescent marker protein or reporter protein used and are known in the art. In one embodiment, CFP (excitation wavelength of about 439 nm and emission wavelength of about 476 nm) is employed. The cells of the invention, which comprise the expression construct of the invention, are particularly suitable for quantitative analysis of the effect of a compound on multipotent stem and progenitor cell differentiation or on totipotent cells. The cells can be counted more accurately due to the fluorescent signal being localized in one spot, i.e. the nucleus, of a cell, as compared to being diffusely expressed in the cytosol of the cell. This causes each “spot” to correspond to a cell, allowing for accurate cell counts that are not easily affected by cellular morphology or interactions with other cells.

Compounds screened or evaluated can be administered or delivered in vivo to the non-human transgenic mammal of the present invention. The compounds can also be studied in vitro. As used herein, the phrases “contacting live multipotent stem and progenitor cells”, “contacting live totipotent stem and progenitor cells” and “contacting live neural stem and progentior cells” with a compound includes in vitro treatment of cells as well as in vivo administration of the compound.

Evaluation of a given compound can be carried out by comparing the measurement of the level of a marker protein or reporter protein in organs or regions thereof in animals who have received the compound in vivo to the marker protein or reporter protein measurement in the corresponding organs or regions thereof in control animals that have not received the compound. Another suitable method of evaluating the effects of compounds administered in vivo includes harvesting and isolating cells from a sacrificed non-human transgenic mammal who had received the compound and comparing the marker protein or reporter protein measurement in the isolated cells to control cells obtained from non-human transgenic mammals who have not received the compound. Compounds administered in vivo and their effects on the cells can be evaluated, for example, by observing tissue fluorescence changes or by FACS of cells harvested from the sacrificed non-human transgenic mammals.

Compounds can also be screened in vitro by employing cells isolated from the non-human transgenic mammal or cells (e.g., totipotent stem and progenitor cells; multipotent stems and progenitor cells) transfected with a construct comprising a promoter sequence, a gene encoding a nuclear localization signal peptide fused to a marker protein or reporter protein and a regulatory sequence present in the second intron of a mammalian nestin gene by the methods described above. The cells can be contacted with a compound to be assessed and the marker protein or reporter protein (e.g. fluorescent protein) in the nuclei of the cells in the presence of the compound is measured and compared to the marker protein or reporter protein measured in control cells. As will be appreciated by those skilled in the art, the sample of cells in the presence of the compound may be matched to the control cell sample in such a manner that any difference in the marker protein or reporter protein measurement (e.g., fluorescence) can be attributed solely to the effect of the compound.

In one embodiment of the invention, live multipotent stem and progenitor cells which have integrated into their genome DNA comprising a regulatory sequence of a mammalian nestin gene operably linked to a gene coding for a nuclear localization signal peptide fused to a marker protein or reporter protein are contacted with a compound to be screened. In the absence of cell destruction, a decrease in the marker protein or reporter protein measurement (e.g., fluorescence) observed in cells exposed to the compound, compared to the measured marker protein or reporter protein of control cells is indicative of the compound's ability to promote (enhance, increase) differentiation of multipotent stem and progenitor cells into cells that no longer express the nestin gene.

A compound's ability to inhibit (decrease) differentiation is indicated by a prolonged measurement of the marker protein or reporter protein. In the embodiment in which the marker protein or reporter protein employed is a fluorescent protein, decreased differentiation of multipotent stem and progenitor cells in the presence of the compound is indicated by prolonged fluorescence in cells in the presence of the compound, as compared to the fluorescence of control cells. In other words, cells in the presence of a compound which inhibits differentiation will fluoresce for a longer period of time than control cells.

In a preferred embodiment of the invention, the isolated cells are neural stem and progenitor cells and a decrease or increase in the marker protein or reporter protein measurement (e.g., fluorescence) observed when these cells are exposed to the compound, as compared to the marker protein or reporter protein measurement (e.g., fluorescence) of control cells, is indicative of the compound's ability to promote or retard differentiation of neural stem and progenitor cells into neurons and glial cells.

In another embodiment of this invention, cells in developmental stages that precede the expression of the nestin gene (e.g. totipotent cells) can be used to screen compounds that promote their differentiation into cells that express the nestin gene, e.g. multipotent stem and progenitor cells. For example, totipotent cell differentiation after exposure to a compound can be assessed for enhanced fluorescence or enhanced presence of another marker protein or reporter protein, as compared to control totipotent cells which have not been contacted or exposed to the compound. In a preferred embodiment, the multipotent stem and progenitor cells include neural stem and progenitor cells.

Another embodiment of this aspect of the invention further comprises measuring an additional reporter or marker expression. In a particular embodiment, the additional marker expression is glial fibrillary acidic protein. In another embodiment, the 5-bromo-2-deoxyuridine (BrdU) incorporation is measured.

Totipotent cells can be isolated from the non-human transgenic mammal, progeny thereof or from a non-human transgenic mammalian embryo of the invention. Examples of techniques for isolating totipotent cells include: culturing embryonic stem (ES) cells, dissociating blastocysts, FACS sorting based on totipotent specific promoter driving the expression of a fluorochrome, totipotent specific cell surface marker selection by antibody, FACS, magnetic bead, affinity columns or antibody affixed to petri dish.

As will be appreciated by those of skill in the art, the control totipotent cells are matched to the totipotent cells contacted with the compound in every other respect except the presence of the compound being assessed. Examples of compounds that can be screened for promoting the differentiation of totipotent cells include those described above. In a preferred embodiment, the compound to be assessed is selected from the group consisting of a growth factor, a neurotrophin, a therapeutic agent, and a diagnostic agent.

Compounds can also be assessed for their toxicity to multipotent stem and progenitor cells, for example to neural stem and progenitor cells. Live cells are contacted with a compound to be assessed and the marker protein or reporter protein measurement (e.g., fluorescence) observed from these cells is compared to the fluorescence of control cells. By itself, a decrease in the measurement (e.g., fluorescence) of cells in the presence of the compound can be indicative of both cell destruction by the compound, as well as cell differentiation to cell types which no longer express nestin. In a preferred embodiment of the invention, cell destruction is measured by any technique that is known to one skilled in the art and which is independent of the technique used to measure the marker or reporter protein expression. For example, if fluorescence is employed as the marker protein or reporter protein measurement, a non-fluorescent technique is used to measure cell destruction. A decrease in the marker protein or reporter protein measurement (e.g., fluorescence), coupled with a reduction in the number of live cells in the cells contacted with a compound being assessed for toxicity, when compared to the fluorescence and the number of live control cells (not contacted with the compound) is indicative of the toxicity of the compound to the multipotent stem and progenitor cells or, in a preferred embodiment, to the neural stem and progenitor cells.

In the examples described below, an approach for the quantitative dissection of the neurogenesis cascade in the DG is presented. This approach can be used to analyze changes induced by a range of stimuli in the adult brain. As an exemplary application of the present invention, the results obtained herein indicate that fluoxetine increases the rate of symmetric divisions of ANP cells and that this increase is later manifested as an increase in the number of new neurons in the DG. Furthermore, they suggest that ANP cells are the sole target of fluoxetine among the neurogenic cells in the postnatal nervous system, and that other drug-induced changes in neurogenesis and the eventual increase in new neurons are the consequences of this initial event. This points to a defined step in the neuronal differentiation cascade affected by fluoxetine and provides a starting point to search for the circuits targeted by fluoxetine and for the molecular mechanisms of fluoxetine-induced signaling in the nervous system. Such example illustrates the utility of the transgenic mice of this invention in providing a more detailed, precise understanding of mechanisms of actions of various compounds on the brain.

The present invention is further illustrated by the following examples which are not intended to be limiting. All references cited herein are incorporated by reference in their entirety.

### EXAMPLES

### Example 1

### Construction of nestin-CFPnuc Expression Vector

Subcloning of the nestin promoter, the poly adenylation sequence from SV40 and the second intron from the nestin gene was carried out essentially as described in U.S. App. Pub. No. 2002/0178460 and in J. L. Mignone et al., J. Comp. Neurol. 469, 311 (2004). Briefly, the SV40 splicing/polyadenylation region was removed from a plasmid bearing the nestin promoter (Zimmerman, L., et al., Neuron, 12, 11-24 (1994)), poly A, and second intron of the nestin gene, by cleavage with the XbaI and BamHI restriction enzymes resulting in a 250 nucleotide base pair band, and was ligated into the pBSM13+ vector (commercially available from Stratagene) at the matching restriction site. AscI restriction sites were added at the XbaI site of this plasmid. The second intron (1.8 kb nucleotides) was obtained from the rat nestin promoter/polyA/second intron plasmid using restriction enzymes BamHI and SmaI, and was then ligated into 3′ to the poly-A-pBSM13+ plasmid at the matching restriction sites. The nestin promoter (5.8 kb nucleotides) from the rat nestin promoter/polyA/second intron plasmid was ligated to the polyA/second intron/pBSM13+ plasmid at the NheI-SaII restriction site, placing the nestin promoter 5′ to the poly-adenylation site. Further, in U.S. App. Pub. No. 2002/0178460, as described briefly below, green fluorescent protein code was inserted into the plasmid by using the NotI restriction site of the plasmid that was changed into an AscI restriction site. The GFP gene was digested using appropriate restriction enzymes to create a 780 bp DNA fragment, and was ligated to the nestin promoter/EGFP/SV40 polyA/second intron/pBSM13+ plasmid at the SaII and AscI sites 3′ to the nestin promoter and 5″ to the polyA site.

The above described nestin-GFP plasmid containing promoter and the second intron of the nestin gene and polyadenylation sequences from simian virus 40, was used to prepare the plasmid of the present invention, by substituting GFP for CFP with nuclear localization domain, to generate nestin-CFPnuc construct. The vector backbone was removed by after SmaI digestion and purification and the resulting 8.8 kb fragment was used for pronuclear injections into the fertilized oocytes from C57BL/6xBalb/cBy hybrid mice.

### Example 2

### Creation of Transgenic Mice

The specific fragment, obtained as described in Example 1 above, was introduced into pronuclei of 500 oocytes of the C57BL/6x BALB/cBy hybrid strain. The injected oocytes were then transferred to 12 pseudo pregnant females. Transgene detection was carried out by PCR analysis of DNA isolated from the tails essentially as described in U.S. App. Pub. No. 2002/0178460. The sequences of the primers used for PCR were CCTCTACAAATGTGTGATGGC (corresponding to the SV40 polyadenylation region) (SEQ ID NO.: 1), and GCGCACCATCTTCTTCAAGGACG (corresponding to the EGFP sequence) (SEQ ID NO.: 2). PCR was performed in 30 μl containing 10% DMS, 2.5 mM MgCl₂, 1× PCR buffer, 0.2 nM of each dNTP, 0.4 μM of each primer and 1 u amplitaq (Boeringer Mannheim). 44 cycles of PCR with an annealing temperature of 55° (30 s) and an extension temperature of 65° (1 min) were used. Several independent lines were generated and two lines were used for the subsequent analysis. They produced identical patterns of CFPnuc expression. Transgenic mice were repeatedly mated with C57BL/6 mice for more than 7 generations.

### Example 3

### Methods of Detection and Sample Preparation

Immunohistochemistry. Tissues were fixed by transcardial perfusion with 30 ml of 4% (w/v) paraformaldehyde in PBS, pH 7.4. The brains were removed, cut longitudinally into two hemispheres and postfixed for 3 hrs at room temperature, then transferred to PBS and kept at 4° C. Serial sagittal sections, 50-μm thick, were cut with a Vibratome 1500 (Vibratome, St. Louis, Mo.). Immunostaining was carried following the standard procedure. The sections were incubated with primary antibodies to the target protein (see the list below) at 4° C. in PBS containing 0.2% Triton-100X and 3% BSA. After thorough washing with PBS, appropriate secondary antibodies (see the list below) were incubated in the same solution for 1 hr at room temperature. After washing with PBS, the sections were mounted on gelatine coated slides with DakoCytomation Fluorescent Mounting Medium (DakoCytomation, Carpinteria, Calif.). For the analysis of 5-bromo-2-deoxyuridine (BrdU) incorporation, sections were treated with 2M HCl for 30 min at 55° C., washed with PBS, treated with 1 mM sodium tetraborate for 10 min at room temperature, and finally washed with PBS. The following antibodies were used: chicken anti-GFP (Aves Laboratories, Tigard, Oreg.) 1:500; guinea pig anti-GFAP (Advanced Immunochemicals, Long Beach, Calif.) 1:500; mouse anti-nestin (Chemicon International, Temecula, Calif.) 1:100; mouse anti-NeuN (Chemicon) 1:800; rabbit anti Dcx (generous gift from Dr. C. A. Walsh, Harvard Medical School) 1:2500; rabbit anti-Prox I (generous gift from Dr. S. J. Pleasure, UCSF) 1:2000; rat anti-BrdU (Serotec, Raleigh, N.C.) 1:400 ; mouse anti-PSA-NCAM (Chemicon) 1:400; AlexaFluor 488 goat anti-chicken (Molecular Probes, Willow Creek Road, Eugene, Oreg.) 1:500; AlexaFluor 594 goat anti-mouse (Molecular Probes) 1:500; AlexaFluor 568 goat anti-rat (Molecular Probes) 1:500; Cy5 goat anti-guinea pig (Jackson Immunoresearch, West Grove, Pa.) 1:500; Cy5 goat anti-rabbit (Jackson Immunoresearch) 1:500; Texas Red donkey anti-chicken (Jackson Immunoresearch) 1:500.

Quantification. Quantitative analysis of cell populations was performed by means of design-based (assumption free, unbiased) stereology. Slices were collected using systematic-random sampling. One brain hemisphere was randomly selected per animal. The hemisphere was sliced sagittaly, in a lateral-to-medial direction, from the beginning until the end of the lateral ventricle, thus including the whole DG. The 50 μm slices, obtained with a 1500 Vibratome (Vibratome, St. Louis, Mo.), were collected in 6 parallel sets (5 in the case of the juvenile mice), each set consisting of 12 slices, each slice 300 μm apart from the next.

For counts in the DG, all the cells of each type described were counted in every slice, excluding those in the uppermost focal plane. The volume of the reference space (of the granule cell layer (GCL)+the subgranular zone (SGZ)) was calculated by the Cavalieri-point method. In the subventricular zone (SVZ), for BrdU counts, all the BrdU stained cells were counted in each slice, excluding those in the uppermost focal plane. For nestin-CFP cells, the two-stage approach was used. At least 10 optical disectors (20×20×20 μm), placed following a systematic-random manner, were applied on each slice. Then the volume of the space reference (of the SVZ) was estimated using the Cavalieri-point method.

In the young animals, all the BrdU cells were counted in each slice, excluding those in the uppermost focal plane. The number of nestin-CFPnuc stained cells, the QNPs, and the ANPs, was estimated using the two-stage approach. At least 5 optical dissectors (20×20×20 μm) placed in a systematic-random manner, were applied to each slice. Then the volume of the space reference (of the SGZ) was estimated using the Cavalieri-point method.

All the imaging and quantification procedures were performed using a Zeiss LSM 510 confocal microscope and its software. Statistical analysis and graphs of the numeric data were obtained using SigmaPlot software (SYSTAT, Port Richmond, Calif.) and JMP (SAS Institute Inc., Cary, N.C.).

### Example 4

### Expression of Nestin Marks Neural Stem and Progenitor Cells

Nestin expression is undetectable in neural cells that have differentiated beyond the initial, proliferative stages. The regulatory elements of the nestin gene direct reporter gene expression to the neuroepithelium of the embryo and to stem and progenitor cells of the adult brain (L. Zimmerman et al., Neuron 12, 11 (1994), A. Kawaguchi et al., Mol. Cell. Neurosci. 17, 259 (2001), K. Sawamoto et al., J. Neurosci. Res. 65, 220 (2001), J. L. Mignone et al., J. Comp. Neurol. 469, 311 (2004)). In the transgenic mice of this invention, the CFPnuc reporter is expressed in the developing nervous system and in the neurogenic areas of the adult brain (the dentate gyrus (DG), subventricular zone (SVZ), rostral migratory stream, and olfactory bulb). Importantly, the distribution of the stem/progenitor cells in the neurogenic areas of these mice can be visualized as a dotted pattern corresponding to the nuclei of these cells.

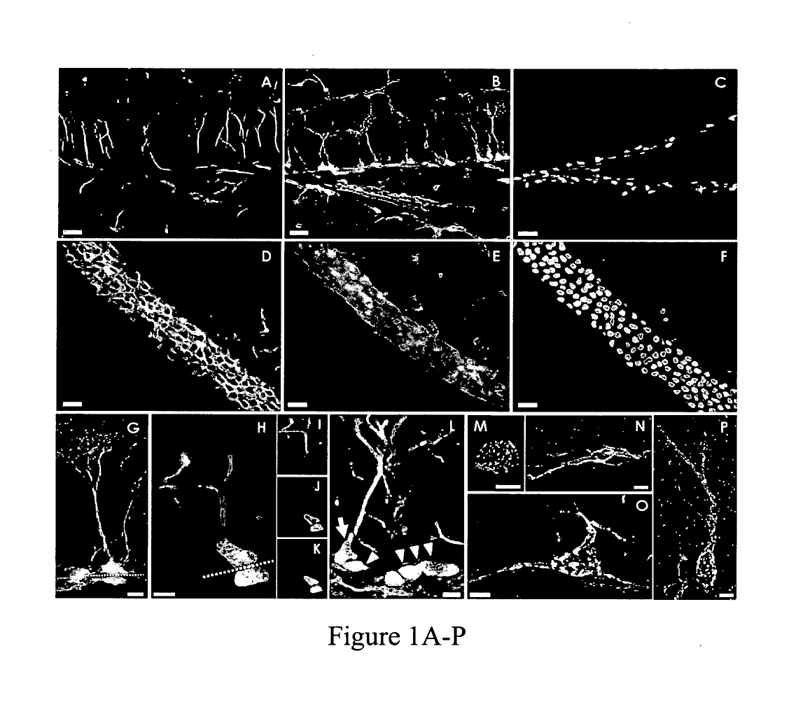

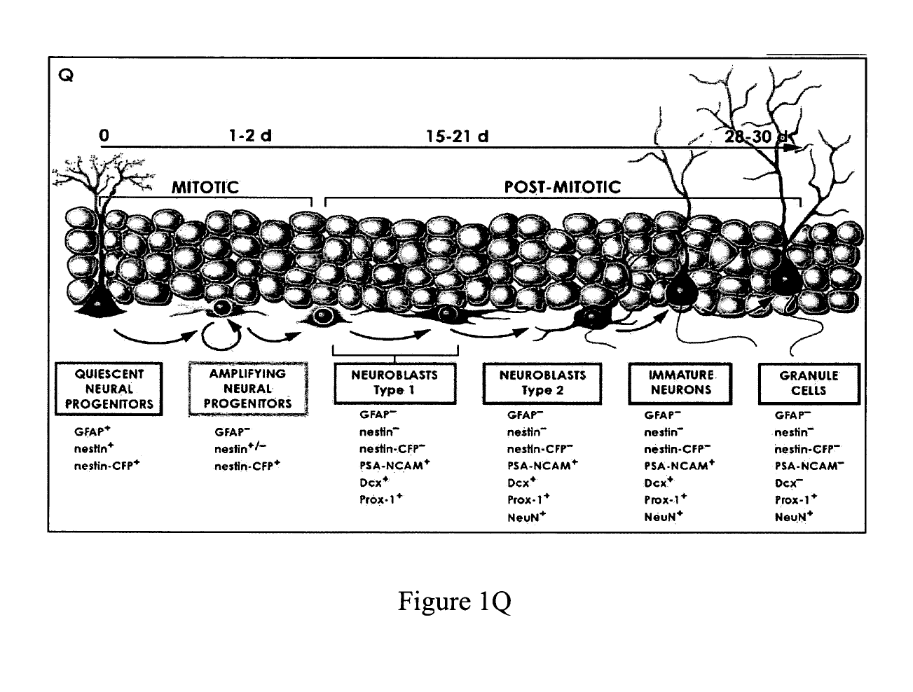

FIG. 1A-F compares the structures of the SVZ and DG as revealed by immunochemistry for nestin and by expression of nestin-CFPnuc or nestin-GFP (Mignone et al. above). Bars are 20 μ in panels A-F, and 5 μ in panels G, H, L-P. Panel A shows the expression of endogenous nestin, detected using a monoclonal antibody against nestin, in the DG of nestin-GFP transgenic mice. The pattern was the same in wild type animals. Panel B shows the expression of GFP in the DG of nestin-GFP transgenic mice, detected using a monoclonal antibody against GFP. Endogenous nestin is seen mostly in the processes (panel A), whereas GFP is present in the processes, the cytoplasm, and the nucleus (panel B). Tight packing of cells in the SGZ prevented accurate enumeration of nestin-GFP expressing cells. Panel C shows the expression of CFPnuc, detected using a polyclonal antibody, in the SGZ of nestin-CFPnuc mice. Transgene-expressing cells were represented by their nuclei, thus making possible accurate cell counts even in densely packed areas. Panels D-F show the expression of nestin (panel D) and GFP (panel E) in the SVZ of nestin-GFP mice, and of CFPnuc (panel F) in the SVZ of nestin-CFPnuc mice. Densely packed SVZ cells, which could not be accurately counted in panel D or E, could be easily quantified in panel F. Although the generation of accurate counts of nestin- or nestin-GFP-positive cells was impossible, unambiguous counts of all of the labeled nuclei in the SVZ and DG of the nestin-CFPnuc mice were generated. This nuclear representation of stem/progenitor cells greatly reduces the complexity of their distribution pattern and permits their unambiguous enumeration (thus capturing the power of BrdU- or thymidine-based enumeration of nuclei).

The discrete steps in the neuronal differentiation cascade in the DG (leading from stem/progenitor cells to differentiated granule neurons) can be easily discerned using the cells from the transgenic mice of the invention, based on the morphology of the cells, the marker proteins that they express, and their mitotic activity (measured by BrdU incorporation). Six classes of cells in the neuronal lineage could be identified in the DG of nestin-CFPnuc mice and are described below. This classification also takes into account the analyses of neuronal precursor populations in the DG described by several groups (G. Kempermann, et al., Trends Neurosci. 27, 447 (2004), B. Seri et al., J. Comp. Neurol. 478, 359 (2004), J. L. Mignone et al. above, D. A. Lim et al., Neuron 28, 713 (2000), V. Filippov et al., Mol. Cell. Neurosci. 23, 373 (2003), S. Fukuda et al., J. Neurosci. 23, 9357 (2003), G. Kronenberg et al., J. Comp. Neurol. 467, 455 (2003)).

The first class is represented by GFAP-positive nestin-CFPnuc cells. The triangular soma and the nuclei of these cells reside in the subgranular zone (SGZ); they extend a single or double apical process radially across the granule cell layer (GCL), terminating as an elaborated arbor of very fine leaf-like processes in the molecular layer. The examples of these cells were observed and were represented in FIG. 1G-K. See also J. L. Mignone above. Panel G shows GFP-expressing neural progenitor cells in the DG of the nestin-GFP mice. The soma of both QNP and ANP cells was seen in the SGZ. QNP cells are characterized by their vertical processes which cross the granule cells layer and end as elaborated arbors in the molecular layer (processes can be also visualized by antibody to GFAP). In contrast, ANP cells are characterized by their lack of the processes. During and immediately after division they could be seen in close contact with QNP; see, for example, a QNP and an ANP cell above and beneath the dashed line in FIG. 1G. Panels H-K show the asymmetric division of stem-like QNP cells in the DG generating ANP cells. Panel H shows that after BrdU labeling, cells with GFAP-labeled processes could be seen dividing (note the horizontal plane of division, dashed line) and generating daughter cells which were deposited below and which did not carry processes or arbors and did not stain for GFAP. Panel H is a composite of three staining shown in Panels I-K, each of which panel shows staining for GFAP (Panel I, showing processes in the upper part of Panel H, stained blue in the fluorescent micrograph), BrdU (Panel J, showing nuclei in Panel H, stained red in the fluorescent micrograph), and CFPnuc (Panel K, stained green in the fluorescent micrograph).

Cells of this class have been described in detail in the above referenced literature, and they correspond to the most primitive, stem-like population in the DG; note, however, that not all of the criteria of stem cells, e.g., ability to self renew, have been demonstrated for these cells (R. M. Seaberg, D. van der Kooy, J Neurosci 22, 1784 (2002) and Trends Neurosci 26, 125 (2003)). Only a small fraction of these cells (less than 2%) can be labeled by BrdU after a short (2 hrs) pulse, indicating their low rate of division and consistent with the quiescent state of these cells; therefore these cells were designated as quiescent neural progenitors (QNP). No instances of symmetric division of such cells (i.e., generating two similar cells or keeping the plane of division perpendicular to the SGZ) were detected; however, these cells can be seen undergoing asymmetric divisions (below).

The second class is represented by small (somatic diameter ˜10 μm) round or oval cells located in the SGZ (FIG. 1H-K). The cells of this class also express nestin-CFPnuc but they do not stain for GFAP and stain very weakly for nestin (this may indicate that CFPnuc protein persists in these cells longer than nestin, or that the nestin is unequally distributed during cell division); they also do not stain for doublecortin (Dcx), PSA-NCAM, or for markers of differentiated neurons. These cells are labeled with BrdU at high frequency (20-25% 2 hours after a single injection of BrdU) indicating that most of them are involved in mitotic activity. These cells were designated as amplifying neural progenitors (ANP). They are often seen in clusters extending along the SGZ, as was observed in FIG. 1L. A QNP cell (arrow) generates an ANP cell (arrowhead) through an asymmetric division, with the plane of division parallel to the SGZ. Nearby was seen a cluster of ANP cells (arrowheads), generated through symmetric divisions in the plane perpendicular to the SGZ. In these figures, GFAP is seen staining processes (red in the fluorescent micrograph) and CFPnuc is seen staining the cell body (green in the fluorescent micrograph), as detected by antibodies. When the plane of division of cells in these clusters was visible, it was most often perpendicular to the SGZ such that the daughter cells remain in the SGZ. Importantly, a fraction of these cells were seen separating from QNPs after mitosis; in each case the division plane was parallel or slightly oblique to the SGZ such that the daughter cell was deposited beneath the QNP cell (FIG. 1H-K) (the plane of division may explain why these cells did not inherit GFAP or nestin which are predominantly localized to the apically positioned processes of the QNPs but not to their soma). Together, these results suggest that QNP cells, as a result of asymmetric divisions, give rise to ANP cells, which then propagate in the SGZ through a series of symmetric divisions.

The third class of precursor cells, still located in the SGZ, can be characterized by their cessation to express nestin or CFPnuc and their expression of Dcx and PSA-NCAM. These cells were designated as Type I neuroblasts (NB1 cells), which start to express markers of young neurons. It can be seen in FIGS. 1M and N that ANPs differentiated into NB1 cells. NB1 cells were still located in the SGZ, ceased to express nestin or nestin-CFPnuc, and started to express PSA-NCAM (green in the fluorescent micrograph), Dcx, and Prox-1. NB1 can be further divided into two subclasses. A small subclass (˜1% of cells in this class) morphologically resembles ANPs, carries short (1-5 μm) horizontal processes, and is the final population in the differentiation cascade that is labeled by BrdU (B. Seri et al., J. Comp. Neurol. 478, 359 (2004)). These cells can be seen in FIG. 1M, as detected by staining with PSA-NCAM antibodies. Most of the cells in this NB1 class are represented by larger (10-15 μm somatic diameter) cells which extend longer (10-30 μm) horizontal processes in the plane of the SGZ and do not incorporate BrdU. These cells can be seen in FIG. 1N, detected by staining with PSA-NCAM antibodies. Thus, the bulk of this class is represented by postmitotic neuronal precursors.

Cells of the fourth class, type 2 neuroblasts or NB2, are larger than NB1 cells (somatic diameter ˜15 μm) and remain confined to the SGZ. They do not express nestin, GFAP, or CFPnuc, and express PSA-NCAM (green in the fluorescent micrograph), Dcx, Prox-1, and NeuN. As can be seen in FIG. 10, detected by staining with PSA-NCAM antibodies, they extend longer (20-40 μm) processes horizontally and obliquely to the plane of the SGZ.

The fifth class of cells corresponds to immature neurons (IN). They express Dcx, PSA-NCAM (green in the fluorescent micrograph), Prox-1, and NeuN. As can be seen in FIG. 1P, detected by staining with PSA-NCAM antibodies, they are larger than the cells of the previous classes (somatic diameter 15-20 μm), and their morphology resembles that of mature granule cells of the DG. Their soma is round or oval and can be found both in the SGZ and, mainly, in the GCL. These cells carry a single apical process that branches in its distal part located in the molecular layer.

The sixth and last class represents differentiated granule neurons, with developed apical dendrites and axons forming the mossy fiber. They cease to express PSA-NCAM and Dcx, but express NeuN and Prox-1.

Thus, using the cells of the invention from the non-human transgenic mammal of the invention, the following observation regarding the neuronal differentiation cascade was made. The differentiation cascade in the DG of nestin-CFPnuc mice can be divided into discrete steps based on the expression of markers, morphology, and mitotic activity. FIG. 1Q shows a schematic summary of the neuronal differentiation cascade in the DG. Quiescent neural progenitors (QNPs) generate, through asymmetric divisions, the amplifying neural progenitors (ANPs) which, after several rounds of symmetric divisions, exit the cell cycle within 1-2 days and become postmitotic type 1 neuroblasts (NB 1 cells). Within the next 15-21 days, NB1 cells mature into type 2 neuroblasts (NB2) and then into immature neurons (IN) with apical processes and basal axons and the soma located in the GCL. After an additional 10-15 days INs acquire the characteristics of mature granule neurons, develop extensive branching, and send long axonal processes forming the mossy fiber. These classes encompass and partially overlap with the subclasses of neuronal precursors defined by other approaches.

### Example 5

### Effect of Fluoxetine

Nestin-CFPnuc reporter transgenic mice of this invention were used to investigate changes in neurogenesis induced by fluoxetine. 7-month old nestin-CFPnuc mice, obtained as described in Example 2, were injected with vehicle (distilled water) or with 10 mg/kg fluoxetine hydrochloride (Tocris, Ellisville, Mo.) once per day for 15 days. On the last day a single injection of BrdU, 150 mg/kg was also administered. For the experiments with juvenile animals, fluoxetine was injected for 15 days starting on postnatal day 5. On the last day they were also given a single injection of BrdU, 150 mg/kg. In both sets of experiments, animals were sacrificed either 24 h or 30 days after the end of the treatment and the BrdU injection.

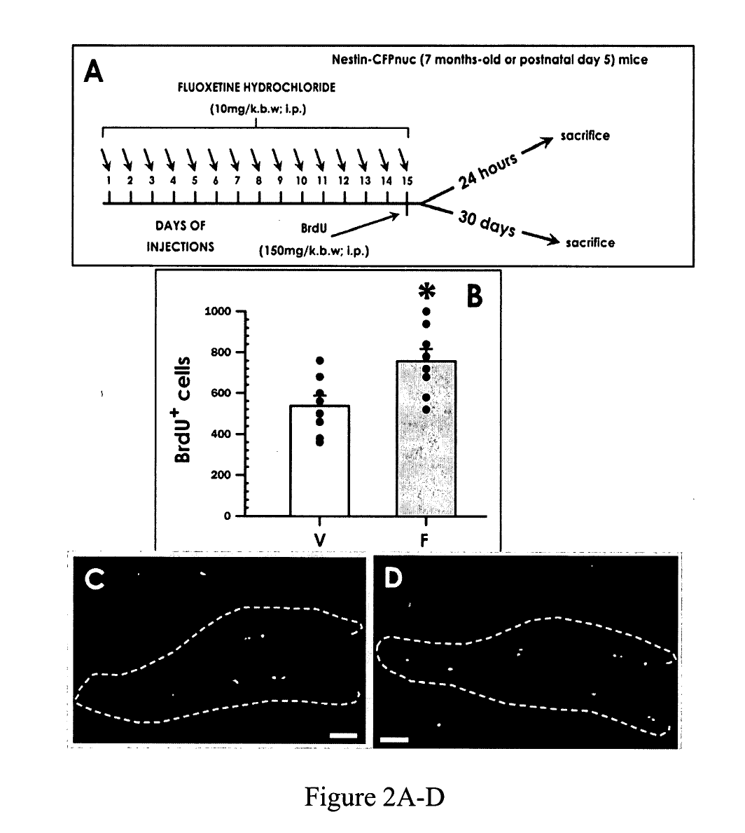

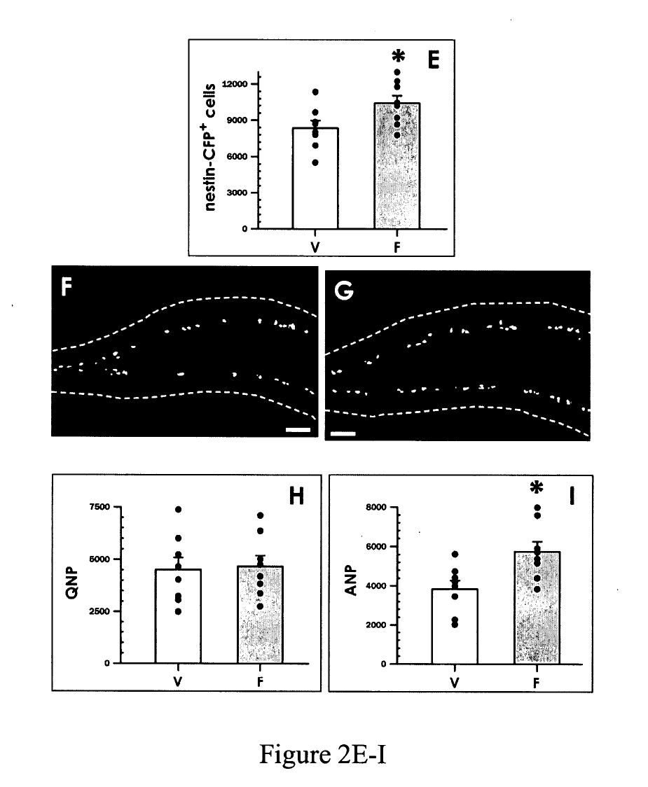

Animals were treated with fluoxetine for 15 days, dividing cells were labeled with BrdU, and selected cell populations in the DG were monitored after 24 hrs using confocal stereology. The experimental paradigm is shown in FIG. 2A. Panels B-I show the results of the chronic administration of fluoxetine. As shown in panel B, fluoxetine increases the number of BrdU-positive cells. Panels C and D show representative photomicrographs, visualized by BrdU-label, of DG sections from animals treated with vehicle (panel C) and fluoxetine (panel D). Dashed line in C, D, F, and G outlines the external limits of the DG. The number of BrdU-labeled cells in the DG was increased by 40.9% (538±51 vs. 758±58, p=0.013) after fluoxetine administration, in line with previous reports on the effects of chronic treatment with the drug (J. E. Malberg et al., J. Neurosci. 20, 9104 (2000), L. Santarelli et al., Science 301, 805 (2003)) (FIG. 2B-D). As shown in Panels E-G, exposure to fluoxetine also increases the number of nestin-CFPnuc cells in the SGZ. Panels F and G show cells expressing CFPnuc. Panel E is the histogram of the results shown in Panels F and G; panel F shows a section of the DG of a control animal; and panel G shows a section of the DG of a fluoxetine-treated animal. The number of CFPnuc-positive cells (i.e., QNPs and ANPs together) increased by 24.7% (8356±622 vs. 10422±646, p=0.037) (FIG. 2E-G). Within total nestin-CFPnuc cells, the number of ANPs (as seen in panel I), but not QNPs (as seen in panel H), increased in response to fluoxetine. Based on expression of GFAP, the QNP class showed no change (4516±582 vs. 4675±518) (FIG. 2H), whereas the number of ANP cells increased by 49.6% (3840±431 vs. 5745±506, p=0.012) (FIG. 21). Bars are 50 μ in panels C, D, F, and G. In all histograms on this and the subsequent figures, white bars correspond to the control injections (V, vehicle), and grey bars to the experimental injections (F, fluoxetine). The results for individual animals (n=8 per group in this figure) are shown as back dots. Error bars show SEM. *p<0.05.

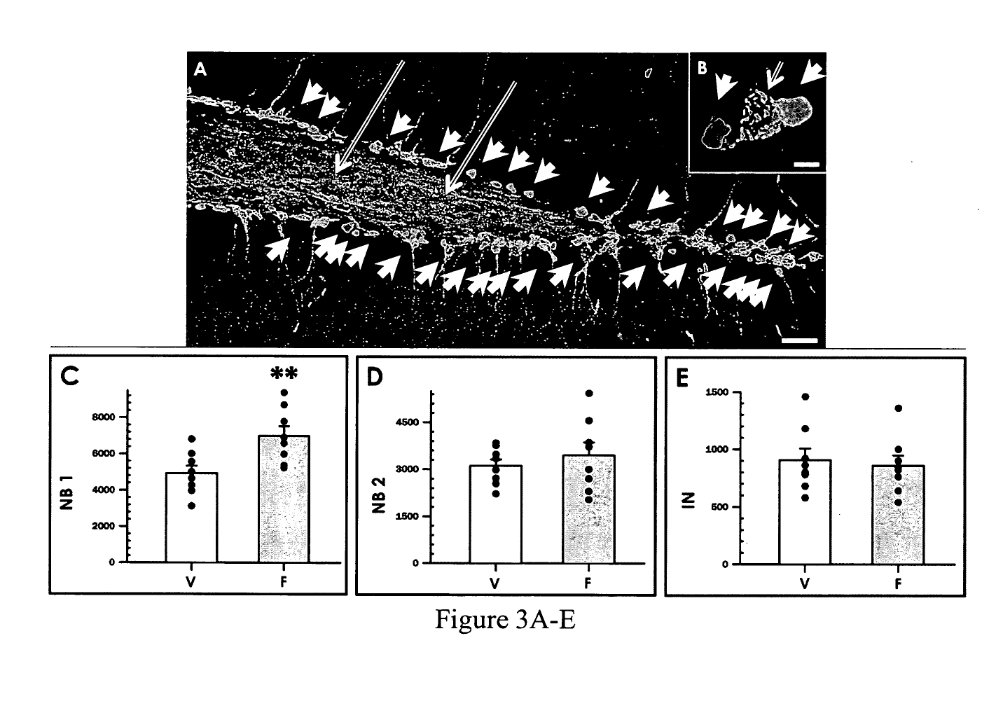

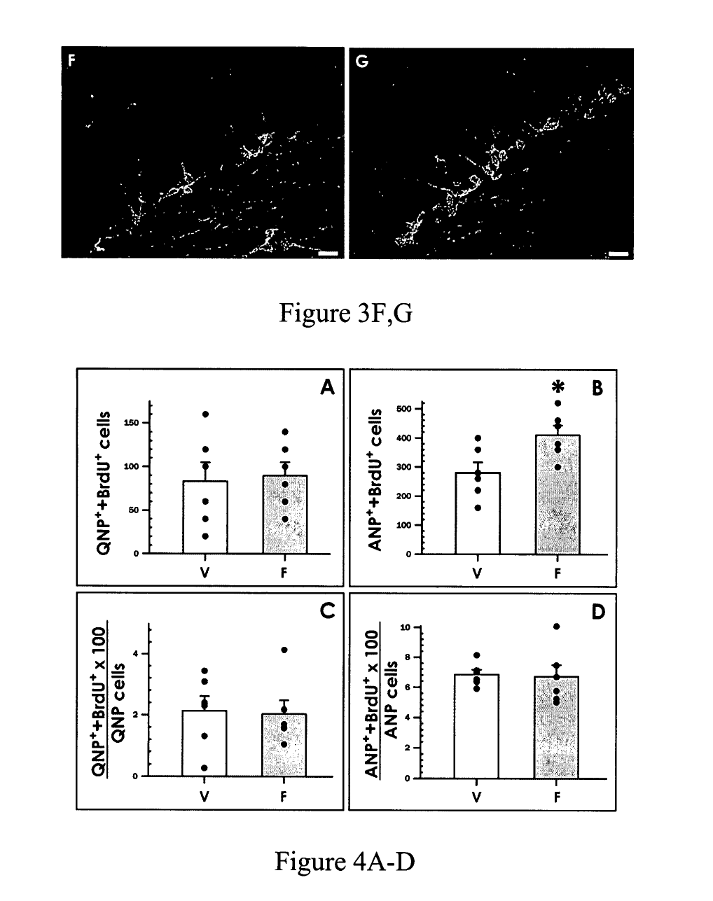

The number of PSA-NCAM-positive cells (which include NB1, NB2, and IN cells, FIG. 3A,B) was increased by 26.5±7.2% (8936±577 vs. 11298±719, p=0.022). In panels A and B, immunostaining for PSA-NCAM (pointed by thin hollow arrows; stained green in the fluorescent micrograph) and nestin-CFPnuc (pointed by thick solid arrows; stained red in the fluorescent micrograph) are shown. Two cell types are distributed throughout the SGZ, often in close apposition to each other; however, they do not overlap. This is illustrated in the inset (PSA-NCAM cell is pointed by a hollow arrow; stained red in the fluorescent micrograph) and nestin-CFPnuc nuclei pointed by thick solid arrows; stained green in the fluorescent micrograph. Colors in the fluorescent micrographs were switched at low magnification for better visualization). Identical changes were seen for Dcx-positive cells. Dcx and PSA-NCAM colocalized in both control and fluoxetine-treated animals, data not shown. When these cells were further subdivided using the criteria described above, postmitotic precursors in the fluoxetine-treated DG of adult mice, analyzed 1 day after BrdU labeling, showed that fluoxetine increases the number of NB1 by 42.1% (4918±418 vs. 6988±538, p=0.089) (FIG. 3C), but not of more advanced NB2 (FIG. 3D) or IN (FIG. 3E) cells (3110±209 vs. 3452±413 and 908±11 vs. 858±88, respectively), compatible with the notion that the wave of increased proliferation-differentiation has not reached those cells classes. In the figures, V stands for vehicle and F for fluoxetine. The results for individual animals (n=8 per group) are shown as back dots. **p<0.01. Panels F and G are representative photomicrographs of DG from control, which was injected with vehicle (panel F), and fluoxetine-treated (panel G) animals. Bars are 20 μm in A, 5 μm in B, and 10 μm in F, G.

### Example 6

### Quantification of ANP Cells and QNP Cells After Fluoxetine Treatment

As indicated above in Example 5, the earliest class affected by fluoxetine is the ANP cells, which are progeny of stem-like QNP cells. Importantly, the QNPs themselves do not increase in number, consistent with the lack of symmetrical divisions in this class. The increase in ANPs can be due to either: a) an increased rate of asymmetric divisions of QNPs (i.e., QNPs may be dividing more often under the influence of fluoxetine, but only give rise to daughter ANP cells while keeping their own number constant), or b) increased symmetric division of ANP cells (i.e., the same number of ANPs may be born from QNPs, but they then divide more frequently). To distinguish between these possibilities, the number of BrdU-labeled QNPs and ANPs were counted.

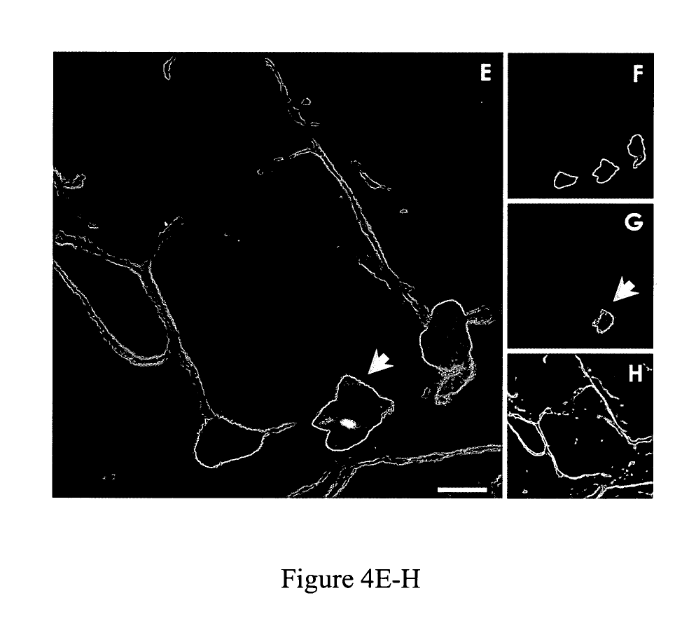

Cells were triple labeled (CFPnuc, BrdU, and GFAP) to discriminate between QNPs and ANPs, and to quantify their mitotic activity (FIG. 4). In the figures, V stands for vehicle and F for fluoxetine. The number of BrdU-labeled QNPs was not affected by fluoxetine treatment (83±22 vs. 90±16, p=0.8) (FIG. 4A), whereas the number of BrdU-labeled ANPs was increased 46.4% (280±36 vs. 410±33, p=0.023) (FIG. 4B). The fraction of dividing cells among QNPs (FIG. C) and ANPs (FIG. 4D) did not change. A cluster of ANP cells between two QNP cells in the DG of a fluoxetine-treated animal. All cells express nestin-CFPnuc (FIG. 4F), but only ANP cells incorporate BrdU (FIG. 4G). QNP cells are identified by the presence of GFAP-positive processes (FIG. 4H). GFAP-staining (blue in the fluorescent micrograph) is shown in Panel H, CFPnuc in Panel F (green in the fluorescent micrograph), and BrdU in Panel G (red in the fluorescent micrograph). Bar is 5 μm in panel E, which is a composite of panels F-H. BrdU staining, colocalizing with nestin-CFPnuc in part and slightly difficult to see, is pointed by a thick arrow. This indicates that the rate of QNP cell division is unchanged and that fluoxetine increases symmetric divisions of ANP cells. When considered together with the data on other cell classes, these results suggest that ANPs are the only class of precursor cells in the DG that directly respond to fluoxetine. The results for individual animals (n=6 per group) are shown as back dots. *p<0.05.

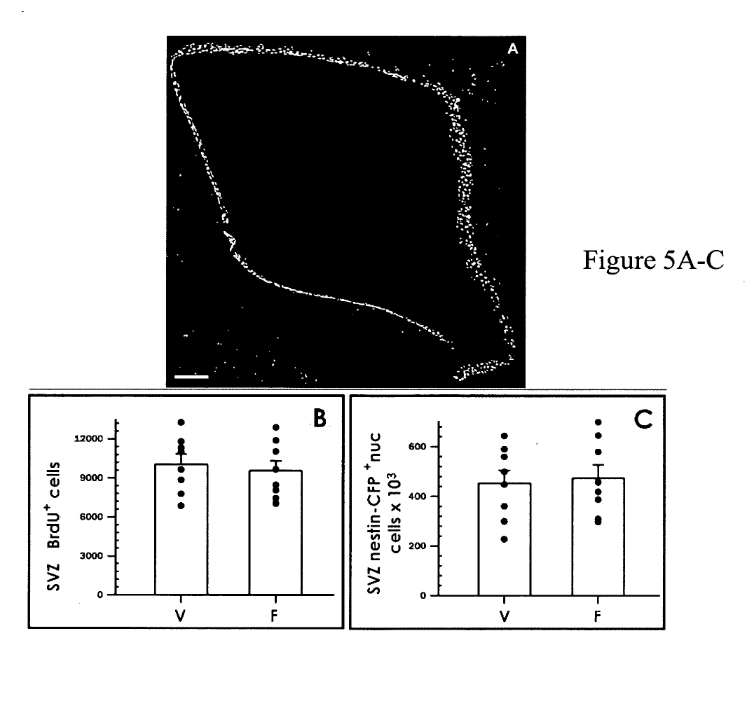

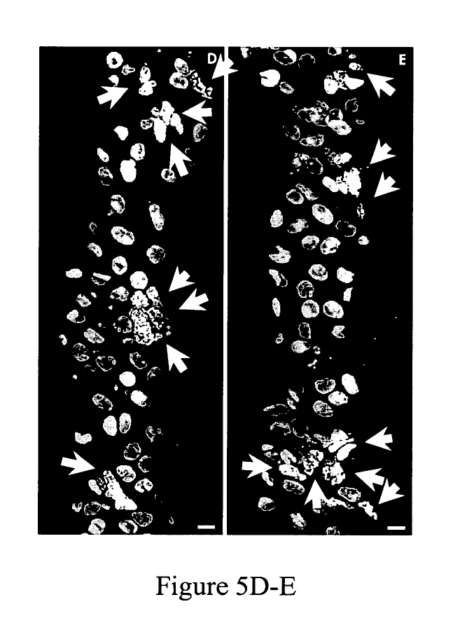

The changes in the SVZ, another major neurogenic region, were also analyzed by observing the SVZ of the lateral ventricle of the nestin-CFPnuc mouse, stained for CFPnuc (FIG. 5A). FIG. 5B shows BrdU-positive cells and FIG. 5C shows nestin-CFPnuc cells. FIG. 5D shows the control animal (injected with vehicle) and FIG. 5E shows fluoxetine-treated animal after staining for CFPnuc (red in the fluorescent micrograph) and BrdU (indicated by thick arrows; green in the fluorescent micrograph). No changes were observed in the number of BrdU-labeled cells (10058±766 vs. 9550±769) (FIG. 5B, D, E), in agreement with the previous observations in rats (J. E. Malberg et al., J. Neurosci. 20, 9104 (2000)) Furthermore, no significant changes were seen either in the number of nestin-CFPnuc cells, (454±52 vs 473±55×10³) (FIG. 5C, D, E), or in their density (648±55 vs. 687±64×10³×mm³), or in the volume (0.648±0.058 vs. 0.603±0.051) of the SVZ. Together, the data indicate that the fluoxetine-induced increase in the number of early progenitor cells is specific for the DG and does not affect the SVZ. Bars are 50 μm in A, and 5 μm in D, E.

### Example 7

### Effect of Fluoxetine on Adult Brain

The CFPnuc transgenic mice were used to show that fluoxetine affects a specific step of this cascade both in the adult and juvenile brain, increasing symmetric divisions of a particular early neural progenitor class in the DG.

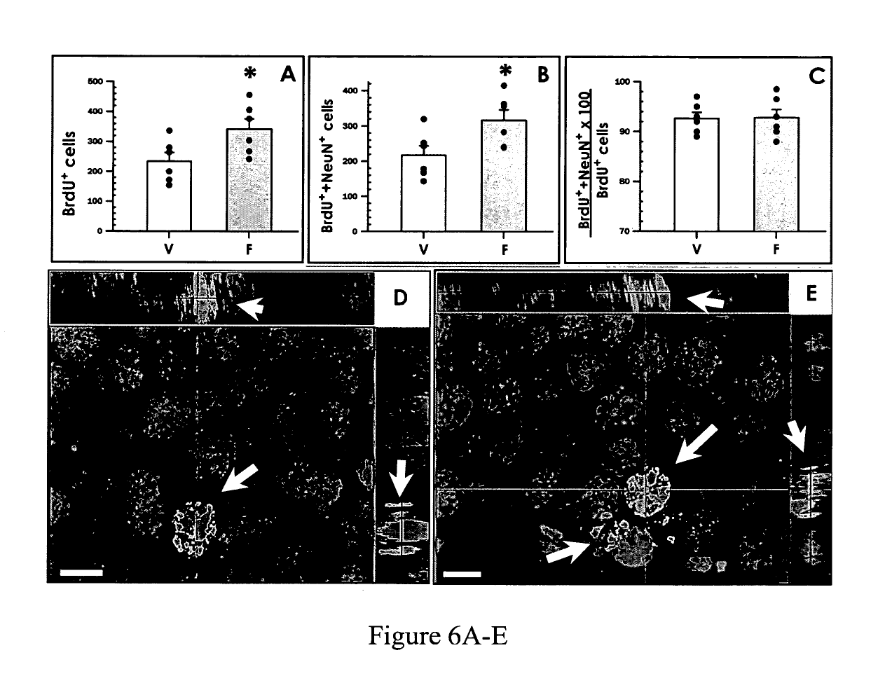

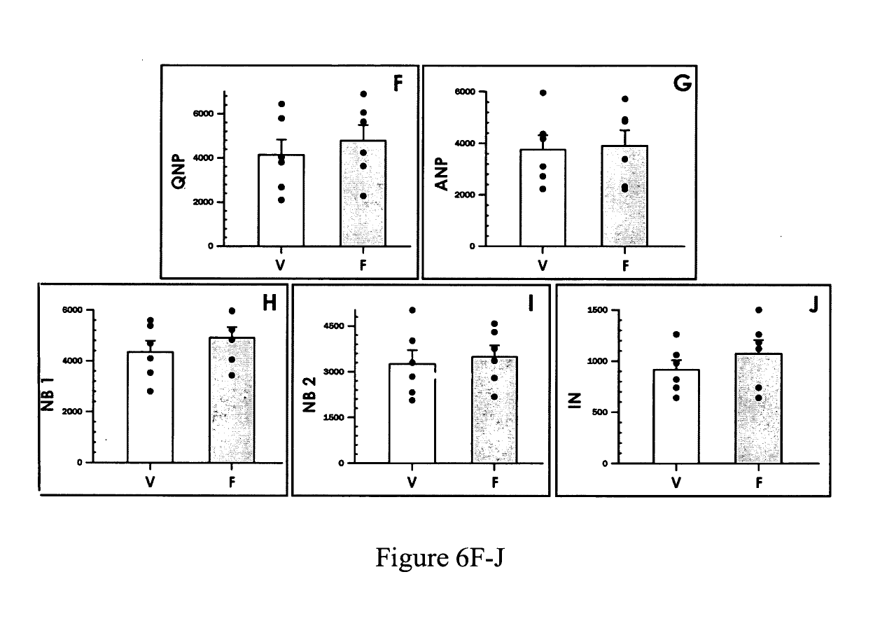

To investigate whether the fluoxetine-induced increase in progenitor cells is later manifested as an increase in the number of new neurons in the DG and whether the increase is maintained after the cessation of treatment with fluoxetine, we performed the fluoxetine treatment and BrdU labeling as described above but sacrificed the animals 30 days (instead of 1 day) later. In this setting, the number of BrdU-labeled cells was 46.2% (234±28 vs. 342±24, p=0.037) higher in the fluoxetine-treated group (FIG. 6A). Fluoxetine also increases the number of BrdU- and NeuN-double-positive cells (FIG. 6B); however, the fraction of such cells among total BrdU-positive cells remained the same (FIG. 6C). Figures D and E are representative photomicrographs of DG from control (injected with vehicle) (D) and fluoxetine-treated (E) animals. These photomicrographs show that new cells choose neuronal fate. The orthogonal projections are shown to confirm double labeling (shown by arrows) throughout the extent of positive cells. Bars are 10 μm in panels D and E. The number of BrdU-labeled NeuN-positive neurons was also higher, by 46.3% (216±26 vs. 316±29, p=0.033), in the fluoxetine group (FIG. 6B, C, D, E). The fraction of BrdU⁺NeuN⁺ cells among total BrdU-positive cells did not change (92.7±1.2 vs. 92.8±1.6%) (FIG. 6C). The high percentage of BrdU-labeled cells also stained for NeuN, indicating the majority of surviving newborn cells in the DG become granule neurons, with or without fluoxetine.

Changes in the defined classes of precursor cells were also examined in mice sacrificed 30 days after the end of the treatment with fluoxetine. Neither the total number of nestin-CFPnuc cells, nor the number of cells in QNP (FIG. 6F), ANP (FIG. 6G), NB1 (FIG. 6H), NB2 (FIG. 61), or IN (FIG. 6J) classes was changed, as indicated in each histogram. In the figures V indicates vehicle, and F, fluoxetine. The results for individual animals (n=6 per group) are shown as back dots. *p<0.05. They suggest that once the exposure to fluoxetine ends, the rate of stem/progenitor cell division returns to its baseline rate. Together, these results suggest that the fluoxetine-induced increase in the number of ANP precursors in the DG later translates into an increase in the number of new neurons. They further suggest that the fate of the newborn cells remains unaltered, i.e., the vast majority of the surplus cells become granule neurons.

### Example 8

### The Effect of Fluoxetine on Juvenile Brain

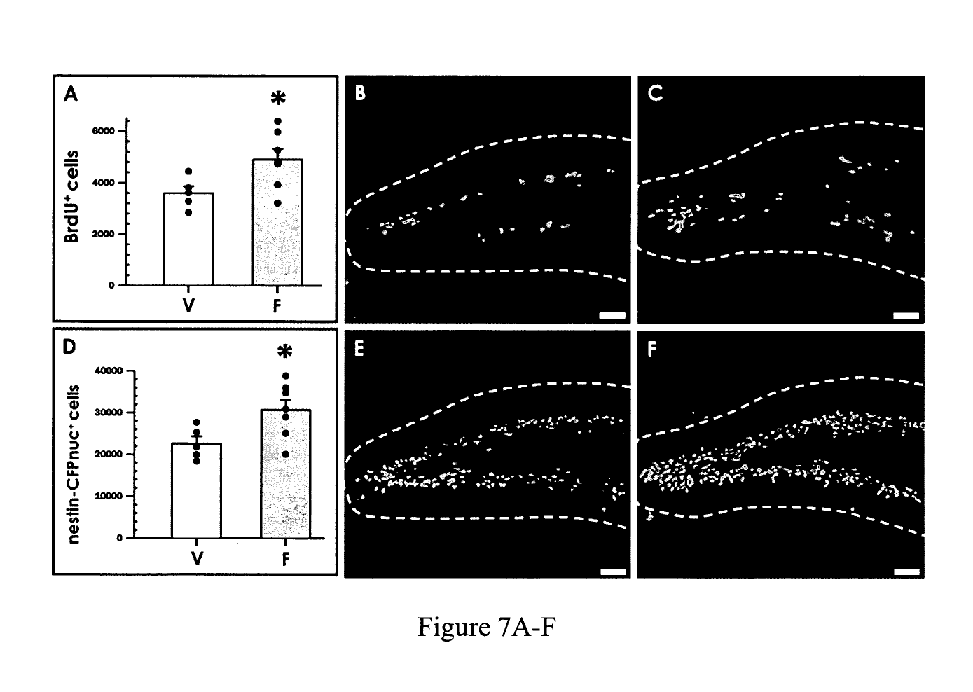

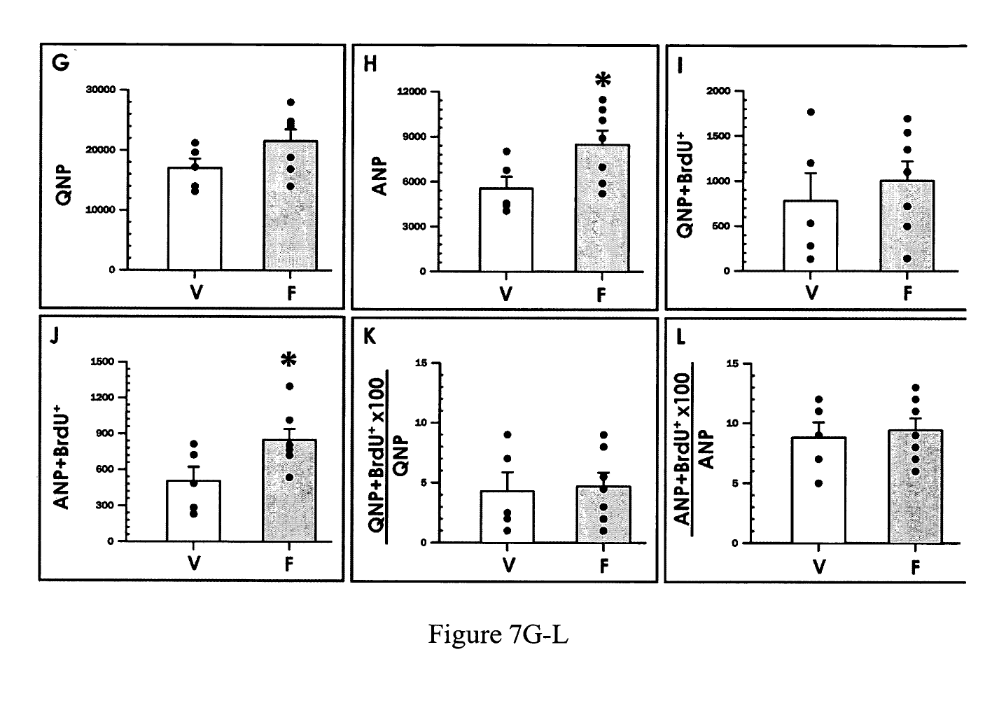

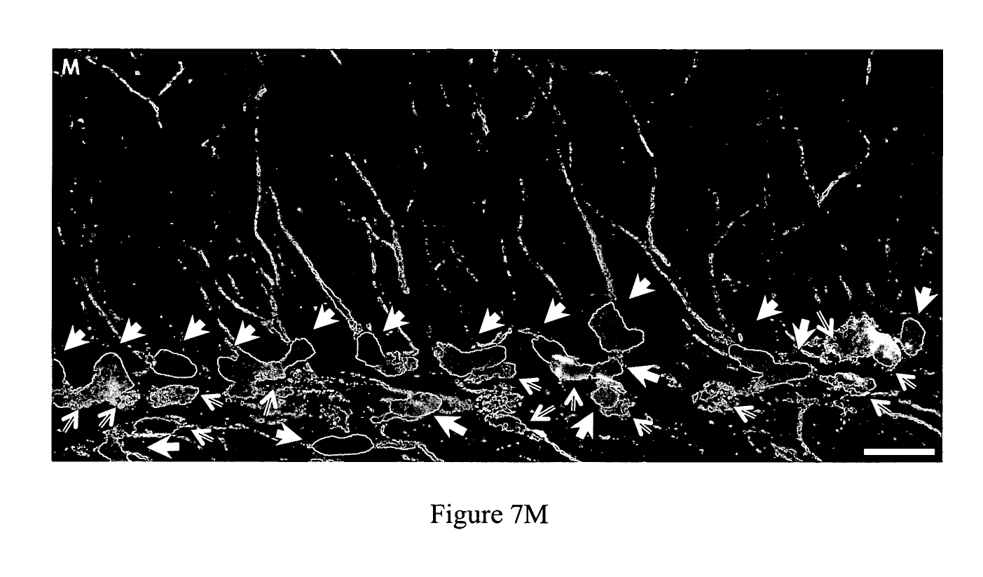

The action of fluoxetine on stem/progenitor cells was investigated for similarity in both the juvenile and adult brain. The same protocol as above (15 days of treatment with fluoxetine followed by labeling with BrdU and analysis 1 day or 30 days later) was used, with the exception that the treatment was started at 5 days of age (p5). In the DG of treated juvenile mice, precursor cells can be divided into the same categories as the cells in the adult brain (note that the animals are 20 or 49 days old at the time of analysis). Furthermore, the fluoxetine-induced changes in the cell subclasses in the juvenile DG are similar to the changes in the adult brain (FIG. 7). After 1 day of BrdU labeling the number of BrdU-positive cells was increased by an extent similar to that in the adult brain, 36.3% (7188±533 vs. 9796±834, p=0.038) (FIG. 7A, B, C). The number of nestin-CFPnuc cells was also increased, 35.6% (45174±3421 vs. 61256±4970, p=0.035) (FIG. 7D, E, F). Panels B, C and E, F show representative photomicrographs of DG sections from animals treated with vehicle (B, E) and fluoxetine (C, D), analyzed for BrdU (B, C) and nestin-CFPnuc (E, F). Dashed line in B, C, E, and F outlines the external limits of the DG. Within these nestin-CFPnuc cells, the change in the number of QNPs did not reach statistical significance (23.5%; 34052±3126 vs. 42048±4328, p=0.134) (FIG. 7G); however, the ANPs responded to the fluoxetine treatment with a significant increase, by 52.6% (11122±1557 vs. 16972±1872, p=0.0475) (FIG. 7H). When the mitotic activity of the DG cells was analyzed by BrdU labeling (FIG. 7M, triple labeling of the DG of a p20 nestin-CFPnuc mouse; GFAP is detected in the processes in the top half of the photomicrograph (blue in the fluorescent micrograph), CFPnuc indicated by thick solid arrows (green in the fluorescent micrograph), and BrdU indicated by hollow arrows (red in the fluorescent micrograph)), the change in the number of dividing QNP cells (28.7%; 1564±614 vs. 2021±434) (FIG. 7I) was not statistically significant, whereas the number of dividing ANP cells strongly increased, by 67.7% (1012±231 vs. 1698±184, p=0.041) (FIG. 7J). As in the adults, the fraction of BrdU⁺NeuN⁺ cells among total BrdU-positive cells did not change (FIG. 7K, BrdU-labeled QNP and FIG. 7L, BrdU-labeled ANP), indicating that, at the time of testing, the rate of division was not affected by fluoxetine.

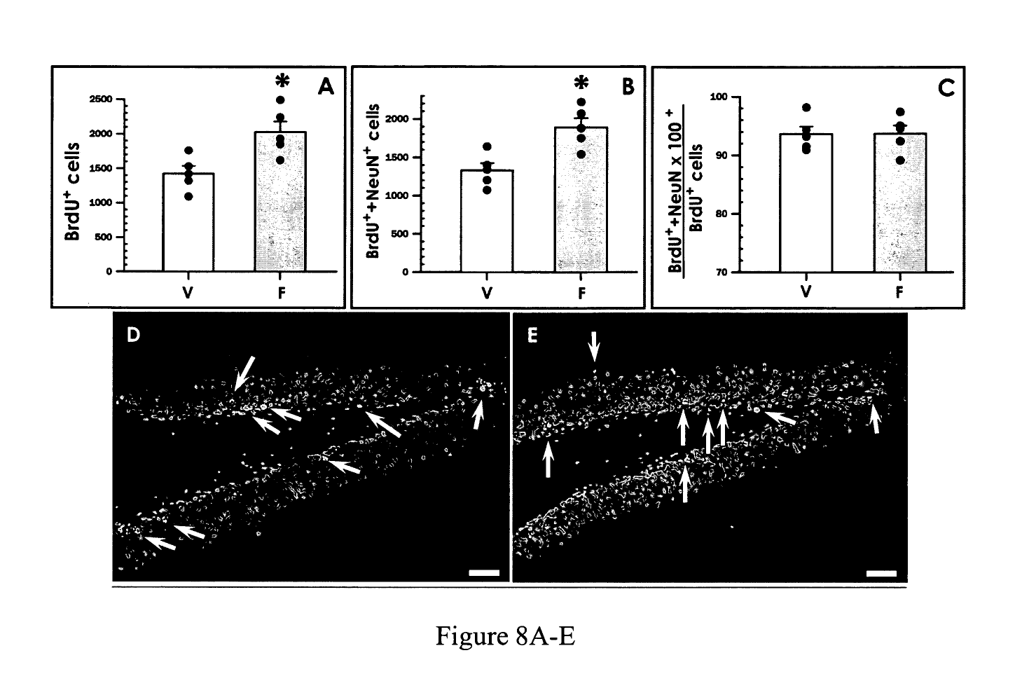

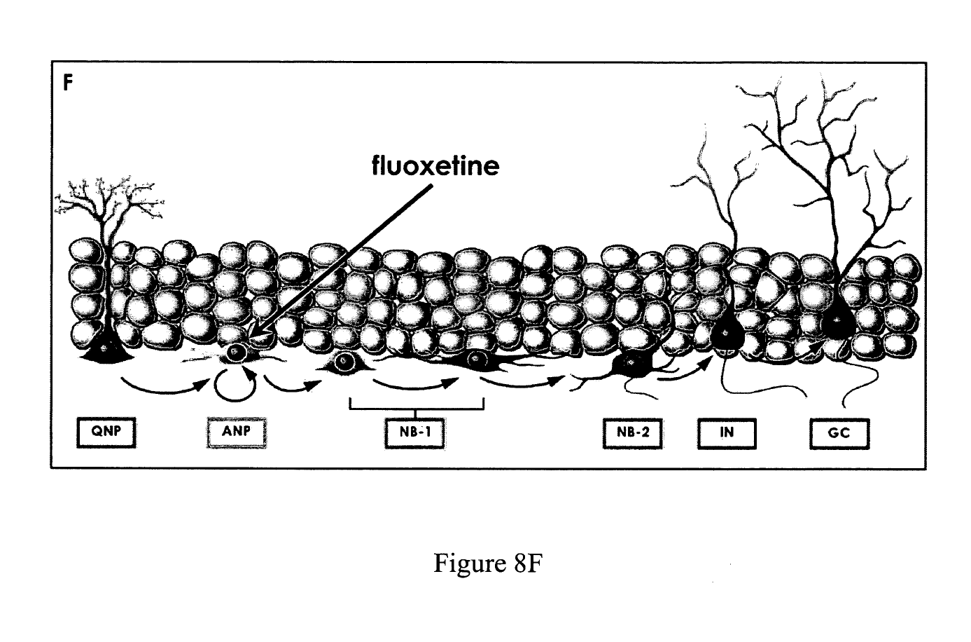

When cells in the DG were analyzed 30 days after the end of fluoxetine administration (FIG. 8), fluoxetine had increased the number of BrdU-positive cells in the DG (FIG. 8A) and the number of BrdU-and NeuN-double-positive cells (FIG. 8B) by 42.2% (1424±111 vs. 2026±153, p=0.013) and 42.3% (1330±96 vs. 1892±119, p=0.006), respectively; however, the fraction of such cells among total NeuN-positive cells remains the same (93.6%) and was as high as in the adult animals. (FIG. 8C). The results can also be seen in representative photomicrographs of DG from control (injected with vehicle) (FIG. 8D) and fluoxetine-treated (FIG. 8E) animals after staining for BrdU (indicated by arrowhead; red in the fluorescent micrograph) and NeuN (overall staining; red in the fluorescent micrograph). Bars are 50 μm. In the figures, V indicates vehicle, and F, fluoxetine. The results for individual animals (n=5 per group) are shown as back dots. *p<0.05.

When the fluoxetine-induced changes were compared across the tested classes of cells in the DG, the interaction between the age and the effect of the drug did not appear to be significant. Thus, at a gross level noticeable differences between the response of specific precursor classes to fluoxetine in juvenile vs. adult mice were detected, in spite of a dramatic overall decline in neurogenesis upon maturation (e.g., note the 7.5-fold and 2.9-fold decrease in QNPs and ANPs respectively). However, it is possible that such differences are more pronounced during the early period of exposure to the drug (note, for instance, a potential trend towards higher activity of QNPs in response to the drug in juveniles), or have a greatly delayed effect on the rate of neurogenesis.

The results obtained herein indicate that fluoxetine increases the rate of symmetric divisions of ANP cells and that this increase is later manifested as an increase in the number of new neurons in the DG. Furthermore, they suggest that ANP cells are the sole target of fluoxetine among the neurogenic cells in the postnatal nervous system (FIG. 8F), and that other drug-induced changes in neurogenesis and the eventual increase in new neurons are the consequences of this initial event. This points to a defined step in the neuronal differentiation cascade affected by fluoxetine and provides a starting point to search for the circuits targeted by fluoxetine and for the molecular mechanisms of fluoxetine-induced signaling in the nervous system. These results also suggest that at the cellular level fluoxetine acts similarly in juvenile and adult brain, lending support to the notion that the basic mechanism of the action of SSRIs is similar in young and adult patients.

1. A non-human transgenic mammal, or progeny or embryo thereof, the genome of which has integrated into it DNA comprising a regulatory sequence of a mammalian nestin gene operably linked to a reporter gene, wherein said reporter gene comprises a DNA sequence of a nuclear localization signal fused in-frame to a DNA sequence encoding a detectable polypeptide, wherein said reporter is expressed and translocated to nuclei in multipotent stem cells and progenitor cells in said transgenic mammal, or progeny or embryo thereof.

2. The non-human transgenic mammal, progeny, or embryo thereof of claim 1, wherein the reporter gene is selectively expressed and translocated to nuclei in multipotent stem cells and progenitor cells in said transgenic mammal, or progeny or embryo thereof.

3. The non-human transgenic mammal, progeny, or embryo thereof of claim 1, wherein the reporter gene is selectively expressed and translocated to nuclei in neural stem cells and neural progenitor cells in said transgenic mammal, or progeny or embryo thereof.

4. The non-human transgenic mammal, progeny, or embryo thereof of claim 1, wherein the mammal is a rodent.

5. The non-human transgenic mammal, progeny, or embryo thereof of claim 4, wherein the rodent is a mouse.

6. The non-human transgenic mammal, progeny, or embryo thereof of claim 1, wherein the regulatory sequence of a mammalian nestin gene is the regulatory sequence of a rat nestin gene.

7. The non-human transgenic mammal, progeny, or embryo thereof of claim 1, wherein the regulatory sequence of a mammalian restin gene comprises the second intron sequence of the mammalian nestin gene.

8. The non-human transgenic mammal, progeny, or embryo thereof of claim 1, wherein the regulatory sequence of a mammalian nestin gene comprises a promoter.

9. The non-human transgenic mammal, progeny, or embryo thereof of any of claims 1 to 8, wherein said detectable polypeptide is a fluorescent protein.

10. The non-human transgenic mammal, progeny, or embryo thereof of claim 9, wherein said detectable polypeptide is a cyan fluorescent protein (CFP).

11. An expression construct comprising a promoter of a mammalian nestin gene operably linked to a reporter gene, wherein said reporter gene comprises a DNA sequence encoding a nuclear localization signal fused in-frame to a DNA sequence encoding a detectable polypeptide.

12. The expression construct of claim 11, further comprising a regulatory sequence found in the second intron of a mammalian nestin gene, which sequence is operably located relative to said promoter and said reporter gene.

13. A cell comprising an expression construct comprising a promoter of a mammalian nestin gene operably linked to a reporter gene, wherein said reporter gene comprises a DNA sequence encoding a nuclear localization signal fused in-frame to a DNA sequence encoding a detectable polypeptide.

14. A method for producing a non-human transgenic mammal comprising the steps of:
a) introducing into a fertilized egg of a non-human mammal DNA comprising a regulatory sequence of a mammalian nestin gene operably linked to a reporter gene, wherein said reporter gene comprises a DNA sequence of a nuclear localization signal fused in-frame to a DNA sequence encoding a detectable polypeptide;
b) introducing said fertilized egg into an oviduct of a non-human mammal of the same species as the source of said fertilized egg to allow said fertilized egg to develop into a viable non-human transgenic mammal; and
c) selecting a non-human transgenic mammal that expresses said reporter, which is translocated to nuclei of multipotent stem cells and progenitor cells in said transgenic mammal.

15. The method of claim 14, wherein the reporter gene is selectively expressed and translocated to nuclei in multipotent stem cells and progenitor cells in said non-human transgenic mammal.

16. The method of claim 14, wherein the reporter gene is selectively expressed and translocated to nuclei in neural stem cells and neural progenitor cells in said non-human transgenic mammal.

17. The method of claim 14, wherein the mammal is a rodent.

18. The method of claim 17, wherein the rodent is a mouse.

19. The method of claim 14, wherein the regulatory sequence of mammalian nestin gene is obtained from rat nestin gene.

20. The method of claim 14, wherein the regulatory sequence comprises the second intron sequence of the mammalian nestin gene.

21. The method of claim 14, wherein the regulatory sequence comprises a promoter.

22. The method of any of claims 14 to 21, wherein said detectable polypeptide is a fluorescent protein.

23. The method of claim 22, wherein the fluorescent protein is a cyan fluorescent protein (CFP).

24. A non-human transgenic mammal produced by the method of claim 14.

25. The non-human transgenic mammal of claim 24, wherein the mammal is a rodent.

26. The non-human transgenic mammal of claim 25, wherein the rodent is a mouse.

27. A method for quantitatively measuring a multipotent stem cell and/or progenitor cell population in a mammalian organ or a region thereof, comprising the step of:
measuring a signal from a detectable reporter expressed in cells from an organ or region thereof of a non-human transgenic mammal, wherein the genome of said mammal has integrated into it DNA comprising a regulatory sequence of a mammalian nestin gene operably linked to said reporter gene, wherein said reporter gene comprises a DNA sequence encoding a nuclear localization signal fused in-frame to a DNA sequence encoding a detectable polypeptide, wherein said reporter is expressed and translocated to nuclei in multipotent stem cells and progenitor cells in said transgenic mammal;
wherein the quantity of said signal correlates with the size of said population.

28. The method of claim 27, wherein said reporter is a fluorescent protein and said signal is fluorescence from the fluorescent protein.

29. The method of claim 28, wherein said fluorescent protein is cyan fluorescent protein.

30. A stem or progenitor cell of a non-human transgenic mammal, the genome of which has integrated into it DNA comprising a regulatory sequence of a mammalian nestin gene operably linked to a reporter gene, wherein said reporter gene comprises a DNA sequence encoding a nuclear localization signal fused in-frame to a DNA sequence encoding a detectable polypeptide, wherein said reporter is expressed and translocated to nuclei in said cell.

31. The stem cell or progenitor cell of claim 30, wherein said cell is a neural stem cell or neural progenitor cell.

32. The stem cell or progenitor cell of claim 30, wherein said cell is a rodent cell.

33. The stem cell or progenitor cell of claim 32, wherein said cell is a mouse cell.

34. The stem cell or progenitor cell of claim 30, wherein said regulatory sequence comprises the second intron sequence of the mammalian nestin gene.

35. The stem cell or progenitor cell of claim 30, wherein said detectable polypeptide is a cyan fluorescent protein.

36. The stem cell or progenitor cell of claim 30, wherein said cell is capable of being affected by administration of fluoxetine when fluoxetine is administered to said mammal.

37. The stem cell or progenitor cell of claim 36, wherein said administration of fluoxetine causes proliferation of said cell.

38. A method for assessing an effect of a compound on proliferation or differentiation of multipotent stem cells or progenitor cells, comprising the steps of:
a) in a test sample, contacting a compound with live multipotent stem cells or progenitor cells the genome of which has integrated into it DNA comprising a regulatory sequence of a mammalian nestin gene operably linked to a reporter gene, wherein said reporter gene comprises a DNA sequence encoding a nuclear localization signal fused in-frame to a DNA sequence encoding a detectable polypeptide, wherein said reporter is expressed and translocated to nuclei in said cell;
b) measuring the value of a signal from said reporter in the test sample; and
c) comparing the value of said signal to that of a control sample;
wherein a difference between the value of the signal from said test sample and that from said control sample indicates that the compound affects proliferation or differentiation of the multipotent stem cells or progenitor cells.

39. The method of claim 38, further comprising the step of measuring expression of an additional reporter or marker, wherein said reporter or marker is not the same as said detectable polypeptide.

40. The method of claim 38, wherein the additional reporter or marker is glial fibrillary acidic protein.

41. The method of claim 38, further comprising the step of measuring cell proliferation.

42. The method of claim 39, wherein the cell proliferation is measured by pulsing with 5-bromo-2-deoxyuridine (BrdU) and detecting cells containing BrdU.

43. A stem or progenitor cell isolated from a non-human mammal, wherein said stem or progenitor cell is capable of being affected by administration of fluoxetine when fluoxetine is administered to said mammal.

44. The stem or progenitor cell of claim 43, wherein said cell is a neural multipotent stem cell or neural progenitor cell.

45. The stem or progenitor cell of claim 43, wherein said administration of fluoxetine causes proliferation of said stem or progenitor cell.

46. A method for treating a disorder comprising the step of administering a pharmaceutical agent to a subject in need thereof, wherein the agent has an effect on a multipotent stem cell or a progenitor cell.

47. The method of claim 46, wherein said multipotent stem cell or a progenitor cell is a neural multipotent stem cell or a neural progenitor cell.

48. The method of claim 46, wherein said effect is a therapeutic or a prophylactic effect.

49. The method of claim 46, wherein said effect is a physiological, morphological, molecular genetic, or biochemical effect.

50. The method of claim 47, wherein said agent is fluoxetine.

51. A method for testing a compound for a physiological, morphological, molecular genetic, or biochemical effect on a multipotent stem cell or a progenitor cell, comprising the steps of:
a) administering said compound to a test mammal which is a transgenic non-human mammal, or progeny or embryo thereof, the genome of which has integrated into it DNA comprising a regulatory sequence of a mammalian nestin gene operably linked to a reporter gene, wherein said reporter gene comprises a DNA sequence of a nuclear localization signal fused in-frame to a DNA sequence encoding a detectable polypeptide, wherein said reporter is expressed and translocated to nuclei in multipotent stem cells and progenitor cells in said transgenic mammal, or progeny or embryo thereof;
b) detecting a change in a physiological, morphological, molecular genetic, or biochemical condition of multipotent stem cells and progenitor cells of the test mammal, wherein such cells are identifiable by the expression of the reporter gene; and
c) comparing said change to a baseline physiological, morphological, molecular genetic, or biochemical condition of corresponding cells in a control mammal that was not administered said compound;
wherein a difference between the changes detected in condition of cells of said test mammal and the change detected in condition of said control mammal indicates that the compound has a physiological, morphological, molecular genetic, or biochemical effect on the multipotent stem cells or progenitor cells.

52. The method of claim 51, wherein said multipotent stem cell or progenitor cell is a neural multipotent stem cell or neural progenitor cell.

53. The method of claim 51, further comprising testing for an effect of said compound on a subpopulation of said multipotent stem cell or progenitor cell, wherein said subpopulation is identified by expression of an additional reporter or marker, wherein said reporter or marker is not the same as said detectable polypeptide.

54. The method of claim 53, wherein the additional reporter or marker is glial fibrillary acidic protein.

55. A method for testing a compound for a physiological, morphological, molecular genetic, or biochemical effect on a multipotent stem cell or a progenitor cell, comprising the steps of:
a) in a test sample, contacting said compound with live multipotent stem cells or progenitor cells the genome of which has integrated into it DNA comprising a regulatory sequence of a mammalian nestin gene operably linked to a reporter gene, wherein said reporter gene comprises a DNA sequence encoding a nuclear localization signal fused in-frame to a DNA sequence encoding a detectable polypeptide, wherein said reporter is expressed and translocated to nuclei in said cell;
b) detecting a change in a physiological, morphological, molecular genetic, or biochemical condition of multipotent stem cells and progenitor cells, wherein said cells are identifiable by the expression of the reporter gene; and
c) comparing said change to a baseline physiological, morphological, molecular genetic, or biochemical condition of corresponding cells in a control sample that was not contacted with said compound;
wherein a difference between the changes detected in condition of cells of said test mammal and the change detected in condition of said control mammal indicates that the compound has a physiological, morphological, molecular genetic, or biochemical effect on the multipotent stem cells or progenitor cells.

56. The method of claim 55, wherein said multipotent stem cell or progenitor cell is a neural multipotent stem cell or neural progenitor cell.

57. The method of claim 55, further comprising the step of testing for an effect of said compound on a subpopulation of said multipotent stem cells or progenitor cells, wherein said subpopulation is identified by expression of an additional reporter or marker, wherein said reporter or marker is not the same as said detectable polypeptide.

58. The method of claim 57, wherein the additional reporter or marker is glial fibrillary acidic protein.


SEQUENCE LISTING (2 sequences)

SEQ ID NO: 1 - DNA, 21 bp, Artificial Sequence
  Description of Artificial Sequence Synthetic primer
cctctacaaatgtgtgatggc

SEQ ID NO: 2 - DNA, 23 bp, Artificial Sequence
  Description of Artificial Sequence Synthetic primer
gcgcaccatcttcttcaaggacg